SEQUENCE LISTING (15 sequences)

SEQ ID NO: 1 - protein, 548 aa, Norovirus
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTISEMSNSRFPVPIDSLHTSPTENIVVQCQNGRVTLDGELMGTTQLLPSQICAFRGTLTRSTSRASDQADTPTPRLFNHRWHIQLDNLNGTPYDPAEDIPAPLGTPDFRGKVFGVASQRNPDSTTRAHEAKVDTTSGRFTPKLGSLEITTESDDFDTNQSTKFTPVGIGVDNEAEFQQWSLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGDSPITVPPNGYFRFESWVNPFYTLAPMGTGNGRRRIQ

SEQ ID NO: 2 - protein, 548 aa, synthetic construct
  source 1..548: note = norovirus chimera GII.3.GII.4F
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTISEMSNSRFPVPIDSLHTSPTENIVVQCQNGRVTLDGELMGTTQLLPSQICAFRGTLTRSTSRASDQADTPTPRLFNHRWHIQLDNLNGTPYDPAEDIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGDSPITVPPNGYFRFESWVNPFYTLAPMGTGNGRRRIQ

SEQ ID NO: 3 - protein, 548 aa, synthetic construct
  source 1..548: note = norovirus chimera GII.3/GII.4IX
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTISEMSNSRFPVPIDSLHTSPTENIVVQCQNGRVTLDGELMGTTQLLPSQICAFRGTLTRSTSRASDQADTPTPRLFNHRWHIQLDNLNGTPYDPAEDIPAPLGTPDFRGKVFGVASQRNPDSTTRAHEAKVDTTSGRFTPKLGSLEITTESDDFDTNQSTKFTPVGIGVDNEAEFQQWSLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGQHDITVPPNGYFRFESWVNPFYTLAPMGTGNGRRRIQ

SEQ ID NO: 4 - protein, 548 aa, synthetic construct
  source 1..548: note = norovirus chimera GII.3/GII.4F+IX
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTISEMSNSRFPVPIDSLHTSPTENIVVQCQNGRVTLDGELMGTTQLLPSQICAFRGTLTRSTSRASDQADTPTPRLFNHRWHIQLDNLNGTPYDPAEDIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGQHDITVPPNGYFRFESWVNPFYTLAPMGTGNGRRRIQ

SEQ ID NO: 5 - protein, 548 aa, synthetic construct
  source 1..548: note = norovirus chimera GII.3/GII.4NERK
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPTENIVVQCQNGRVTLDGELMGTTQLLPSQICAFRGTLTRSTSRASDQADTPTPRLFNHRWHIQLDNLNGTNYDPAEEIPAPLGTPDFRGKVFGVASQRNPDSTTRAHEAKVDTTSGRFTPKLGSLEITTESDDFDTNQSTKFTPVGIGVDNEAEFQQWSLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGDSPITVPPNGYFRFESWVNPFYTLAPMGTGNGRRRIQ

SEQ ID NO: 6 - protein, 548 aa, synthetic construct
  source 1..548: note = norovirus chimera GII.3/GII.4NERK+F
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPTENIVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGDSPITVPPNGYFRFESWVNPFYTLAPMGTGNGRRRIQ

SEQ ID NO: 7 - protein, 548 aa, synthetic construct
  source 1..548: note = norovirus chimera GII.3/GII.4NERK+I
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPTENIVVQCQNGRVTLDGELMGTTQLLPSQICAFRGTLTRSTSRASDQADTPTPRLFNHRWHIQLDNLNGTNYDPAEEIPAPLGTPDFRGKVFGVASQRNPDSTTRAHEAKVDTTSGRFTPKLGSLEITTESDDFDTNQSTKFTPVGIGVDNEAEFQQWSLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGQHDITVPPNGYFRFESWVNPFYTLAPMGTGNGRRRIQ

SEQ ID NO: 8 - protein, 548 aa, synthetic construct
  source 1..548: note = norovirus chimera GII.3/GII.4NERK+F+I
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPTENIVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGQHDITVPPNGYFRFESWVNPFYTLAPMGTGNGRRRIQ

SEQ ID NO: 9 - protein, 548 aa, synthetic construct
  source 1..548: note = norovirus chimera GII.3/GII.4 P2
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTISEMSNSRFPVPIDSLHTSPSGAFVVQCQNGRVTLDGELMGTTQLLPSQICAFRGTLTRSTSRASDQADTPTPRLFNHRWHIQLDNLNGTPYDPAEDIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGDSPITVPPNGYFRFESWVNPFYTLAPMGTGNGRRRIQ

SEQ ID NO: 10 - protein, 548 aa, synthetic construct
  source 1..548: note = norovirus chimera GII.3/GII.4 P3
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPTENIVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEPQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAHTGQHDITVPPNGYFRFESWVNPFYTLAPMGTGNGRRRIQ

SEQ ID NO: 11 - protein, 548 aa, synthetic construct
  source 1..548: note = norovirus chimera GII.3/GII.4 P4
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPSGAFVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEPQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAHTGQHDITVPPNGYFRFESWVNPFYTLAPMGTGNGRRRIQ

SEQ ID NO: 12 - protein, 548 aa, synthetic construct
  source 1..548: note = norovirus chimera GII.3/GII.4 P5
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPSGAFVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGDSPITVPPNGYFRFESWVNPFYTLAPMGTGNGRRRIQ

SEQ ID NO: 13 - protein, 548 aa, synthetic construct
  source 1..548: note = norovirus chimera GII.3/GII.4 P6
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIDSLHTSPSGAFVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGQHDITVPPNGYFRFESWVNPFYTLAPMGTGNGRRRIQ

SEQ ID NO: 14 - protein, 548 aa, synthetic construct
  source 1..548: note = norovirus chimera GII.3/GII.4 P7
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTISEMSNSRFPVPIDSLHTSPSGAFVVQCQNGRVTLDGELMGTTQLLPSQICAFRGTLTRSTSRASDQADTPTPRLFNHRWHIQLDNLNGTPYDPAEDIPAPLGTPDFVGKVFGVASQRNPDSTTRAHEAKVDTTSGRFTPKLGSLEITTESDDFDTNQSTKFTPVGIGVDNEAEFQQWVLPNYSGQFTHNMNLAPAVAPNFPGEQLLFFRSQLPSSGGWSNGVLDCLVPQEWVQHFYQESAPAQTQVALVRYVNPDTGRVLFEAKLHKLGFMTIAKNGQHDITVPPNGYFRFESWVNPFYTLAPMGTGNGRRRIQ

SEQ ID NO: 15 - protein, 548 aa, synthetic construct
  source 1..548: note = norovirus chimera GII.3/GII.4 P8
MKMASNDAAPSNDGAAGLVPEINNEAMALDPVAGAAIAAPLTGQQNIIDPWIMNNFVQAPGGEFTVSPRNSPGEVLLNLELGPEINPYLAHLARMYNGYAGGFEVQVVLAGNAFTAGKVIFAAIPPNFPIDNLSAAQITMCPHVIVDVRQLEPINLPMPDVRNTFFHYNQDSDSRLRLIAMLYTPLRANNSGDDVFTVSCRVLTRPSPDFSFNFLVPPTVESKTKLFTLPILTVSEMSNSRFPVPIEKVFTGPSGAFVVQCQNGRVTLDGELMGTTQLLPSQICAFRGTLTRSTSRASDQADTPTPRLFNHRWHIQLDNLNGTNYDPAEEIPAPLGTPDFVGKVFGVASQRNPDSTTRAHEAKVDTTSGRFTPKLGSLEITTESDDFDTNQSTKFTPVGIGVDNEAEPQQWVLPNYSGQFTHNMNLAPAVAPNFPGEQLLFFRSTLPSSGGWSNMNLDCLVPQEWVQHFYQESAPAQTQVALVRYVNPDTGRVLFEAKLHKLGFMTIAHTGQHDLVIPPNGYFRFESWVNPFYTLAPMGTGNGRRRIQ

(19) United States
(12) Patent Application Publication
(10) Pub. No.: US 2025/0002539 A1
(43) Pub. Date: Jan. 2, 2025

(54) METHODS AND COMPOSITIONS FOR NOROVIRUS CHIMERIC THERAPEUTICS

(71) Applicant: The University of North Carolina at Chapel Hill, Chapel Hill, NC (US)

(72) Inventors: Ralph Steven Baric, Haw River, NC (US); Lisa Chon Lindesmith, Apex, NC (US)

(21) Appl. No.: 18/292,199

(22) PCT Filed: Jul. 25, 2022
(86) PCT No.: PCT/US2022/074103

§ 371 Date: Jan. 25, 2024

Related U.S. Application Data
(60) Provisional application No. 63/225,830, filed on Jul. 26, 2021.

(51) Int. Cl. C07K 14/005; A61K 39/00; A61P 31/14

(52) U.S. Cl. CPC C07K 14/005; A61P 31/14; A61K 2039/5258; A61K 2039/53; A61K 2039/575; C12N 2770/16022; C12N 2770/16034

(57) ABSTRACT

The present invention is directed to methods and compositions for GII.3/GII.4 chimeric norovirus capsid proteins comprising one or more amino acid substitutions. Additionally provided are synthetic backbone molecules, norovirus P particles, synthetic nanoparticles, scaffold immunogens, nucleic acids, mimitopes, polypeptides, virus replicon particles (VRPs), virus like particles (VLPs), vectors, cells, and compositions comprising the same.

### STATEMENT OF GOVERNMENT SUPPORT

This invention was made with government support under Grant Nos. AI109761 and AI1148260, awarded by the National Institutes of Health. The government has certain rights in the invention.

### STATEMENT REGARDING ELECTRONIC FILING OF A SEQUENCE LISTING

A Sequence Listing in XML format, submitted in accordance with 37 C.F.R. § 1.831-1.834, entitled 5470-906WO.xml, 24,944 bytes in size, generated on Jul. 25, 2022 and filed electronically, is provided in lieu of a paper copy. This Sequence Listing is hereby incorporated herein by reference into the specification for its disclosures.

### FIELD OF THE INVENTION

This invention is directed to methods and compositions for norovirus therapeutics, such as vaccines, and diagnostics.

### BACKGROUND OF THE INVENTION

Human noroviruses (HuNoV) cause ˜85-96% of acute non-bacterial gastroenteritis, worldwide. Outbreaks occur in communities, families, recreational facilities, retirement communities, day care centers, schools, cruise/military ships, organ-transplant recipients, hospitals and in the military. HuNoV infection results in ˜200,000 deaths/year, mostly in infants, elderly and the immunosuppressed. With the advent of effective rotavirus vaccination programs, HuNoV have become the major cause of severe gastroenteritis in children and infants, who require hospitalization. As HuNoV are very stable in the environment and highly infectious, effective HuNoV vaccines and immunotherapeutics are key to controlling disease severity in human populations.

The present invention overcomes previous shortcomings in the art by providing methods and compositions for norovirus therapeutics, including multivalent vaccines, and diagnostics.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one embodiment, the present invention provides a chimeric norovirus capsid protein comprising one or more (e.g., two, three, four, etc.) of the following amino acid substitutions in any combination, wherein the numbering is based on the reference amino acid sequence of a norovirus capsid protein of a norovirus strain GII.3 identified as SEQ ID NO:1: R341V, S412V, D512Q, S513H, P514D, I234V, P324N, and/or D330E.

In another embodiment, the present invention provides a chimeric norovirus capsid protein comprising one or more (e.g., two, three, four, etc.) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); and/or c) amino acid residues I234V, P324N, and D330E (NERK).

In another embodiment, the present invention provides a chimeric norovirus capsid protein further comprising one or more of the following amino acid substitutions in any combination, wherein the numbering is based on the reference amino acid sequence of a norovirus strain GII.3 identified as SEQ ID NO:1: T254S, E255G, N256A, I257F, F408P, K509H, and/or N510T.

In another embodiment, the present invention provides a synthetic backbone molecule comprising a set of amino acid residues that form a norovirus conformation epitope, comprising the following set of amino acid substitutions, wherein the numbering is based on the reference amino acid sequence of a norovirus capsid protein of a norovirus strain GII.3 identified as SEQ ID NO:1 a) amino acid residues R341V and S412V (Epitope F); b) amino acid residues D512Q, S513H, and P514D (Epitope I); and/or c) amino acid residues I234V, P324N, and D330E (NERK), wherein the synthetic backbone molecule is not a norovirus capsid protein. In some embodiments, the present invention provides a synthetic backbone molecule further comprising one or more of the following amino acid substitutions in any combination: T254S, E255G, N256A, I257F, F408P, K509H, and/or N510T.

In another embodiment, the present invention provides a norovirus P particle comprising a set of amino acid residues that form a norovirus conformation epitope, comprising one or more (e.g., two, three, four, etc.) of the following amino acid substitutions in any combination, wherein the numbering is based on the reference amino acid sequence of a norovirus capsid protein of a norovirus strain GII.3 identified as SEQ ID NO:1: a) amino acid residues R341V and S412V (Epitope F); b) amino acid residues D512Q, S513H, and P514D (Epitope I); and/or c) amino acid residues I234V, P324N, and D330E (NERK), wherein the synthetic backbone molecule is not a norovirus capsid protein. In another embodiment, the present invention provides a norovirus P particle further comprising one or more of the following amino acid substitutions in any combination: T254S, E255G, N256A, I257F, F408P, K509H, and/or N510T.

In another embodiment, the present invention provides a synthetic nanoparticle and/or scaffold immunogen comprising amino acid residues 224 through 529 of the chimeric norovirus capsid protein of the present invention, wherein the numbering is based on the reference amino acid sequence of a norovirus capsid protein of a norovirus strain GII.3 identified as SEQ ID NO:1. In another embodiment, the present invention provides a synthetic nanoparticle and/or scaffold immunogen further comprising one or more of the following amino acid substitutions in any combination: T254S, E255G, N256A, I257F, F408P, K509H, and/or N510T.

Additionally provided herein are isolated nucleic acid molecules, virus replicon particles (VRP), vectors, virus like particles (VLP), and pharmaceutical compositions comprising a chimeric norovirus capsid protein or norovirus P particle of the present invention.

Also provided herein is a method of producing an immune response to a norovirus virus in a subject, comprising administering to the subject an effective amount of a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention.

Also provided herein is a method of treating a norovirus virus infection in a subject, comprising administering to the subject an effective amount of a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention.

Also provided herein is a method of preventing a disorder associated with norovirus infection in a subject, comprising administering to the subject an effective amount of a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention.

Also provided herein is a method of reducing the risk of developing a disorder associated with norovirus infection in a subject, comprising administering to the subject an effective amount of a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention.

Also provided herein is a method of protecting a subject from the effects of norovirus infection, comprising administering to the subject an effective amount of a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention.

The present invention also provides diagnostic methods. Thus, also provided herein is method of detecting a neutralizing antibody to a norovirus, the method comprising determining whether an antibody binds to a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention, wherein binding by the antibody to the chimeric norovirus capsid protein and/or synthetic backbone and/or P particle and/or synthetic nanoparticle and/or scaffold immunogen indicates that the antibody is a neutralizing antibody to a norovirus.

Also provided herein is a method of identifying a neutralizing antibody to a norovirus, comprising: (a) contacting an antibody with a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention; and (b) determining if the antibody binds to the chimeric norovirus capsid protein and/or synthetic backbone and/or P particle and/or synthetic nanoparticle and/or scaffold immunogen, wherein binding by the antibody to the chimeric norovirus capsid protein and/or synthetic backbone and/or P particle and/or synthetic nanoparticle and/or scaffold immunogen identifies the antibody as a neutralizing antibody to a norovirus.

Also provided herein is a method of identifying an immunogenic composition that induces a neutralizing antibody to a norovirus in a subject, the method comprising: (a) contacting a biological sample from a subject that has been administered the immunogenic composition with a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention; (b) determining if the biological sample comprises an antibody that binds the chimeric norovirus capsid protein and/or synthetic backbone and/or P particle and/or synthetic nanoparticle and/or scaffold immunogen; and (c) identifying the immunogenic composition as inducing a neutralizing antibody to a norovirus in the subject if the biological sample comprises an antibody that binds to the chimeric norovirus capsid protein and/or synthetic backbone and/or P particle and/or synthetic nanoparticle and/or scaffold immunogen.

Also provided herein is a method of identifying an immunogenic composition that induces a neutralizing antibody to a norovirus in a subject, the method comprising: (a) administering an immunogenic composition comprising a norovirus antigen to a subject in an amount effective to induce antibodies against the norovirus antigen; (b) contacting a biological sample from the subject with a chimeric norovirus capsid protein of the present invention, a synthetic backbone molecule of the present invention, a norovirus P particle of the present invention, a synthetic nanoparticle and/or scaffold immunogen of the present invention, a nucleic acid molecule of the present invention, a VRP of the present invention, a vector of the present invention, a VLP of the present invention, and/or a composition of the present invention; (c) determining if the biological sample comprises an antibody that binds the chimeric norovirus capsid protein and/or synthetic backbone and/or P particle and/or synthetic nanoparticle and/or scaffold immunogen; and (d) identifying the immunogenic composition as inducing a neutralizing antibody to a norovirus in the subject if the biological sample comprises an antibody that binds the chimeric norovirus capsid protein and/or synthetic backbone and/or P particle and/or synthetic nanoparticle and/or scaffold immunogen.

### BRIEF DESCRIPTION OF THE DRAWINGS

FIG. 1 shows a representation of the human norovirus GII.4 capsid surface P domain, formed of dimers of the capsid protein, with labeled epitopes A, D, E, F, I, and the antibody access regulating (NERK, breathing core) domain.

FIG. 2 shows a representation of the human norovirus GII.3 capsid surface P domain, formed of dimers of the capsid protein, with highlighted residues (black) of the NERK, F, and I epitopes.

FIGS. 3A-3B show representations of the human norovirus GII.3 capsid surface P domain with mapping of GII.4 epitopes onto the monomer of the GII.3 P dimer (FIG. 3A) and comparing the sequence divergences between GII.3 and GII.4 2002 (FIG. 3B, light grey=conserved (no change), dark grey=conserved changes, black=non-conserved changes); locations of further substitutions made for GII.3/GII.4 P2 (small dash circle), P3 (large dash circle), and P4 (solid circle) chimeric VLPs.

FIGS. 4A-4B show GII.4 antigenic region (F+I and NERK+F+I) transplanted into GII.3 backbone improved the neutralizing potency of serum compared to GII.3 (FIG. 4A), and the fold increase in GII.3 neutralizing antibody potency compared across the GII.3/GII.4 chimeric antigens (FIG. 4B). NERK, F+I and NERK+F+I transplantation into GII.3 improved serum blockade potency. * significantly different from GII.3 (ANOVA with Dunn's multiple comparison test). Lines=geometric mean, error bars=95% confidence intervals. Dashed lines=assay limit of detection. Each marker represents an adult serum sample.

FIG. 5 shows a representation of the human norovirus GII.3 capsid surface P domain with additional regions of interest circled and identified with arrows.

FIGS. 6A-6B show a schematic of experimental design (FIG. 6A) and a phylogenetic tree of identified GII.4 norovirus strains (FIG. 6B), including example variants tested.

FIGS. 7A-7D show data plots regarding GII chimeric VLP booster post mRNA vaccination regimen. The booster post mRNA vaccination broadens neutralizing antibody (nAb) responses to the mRNA vaccine components, additional genotypes, and antigenically divergent GII.4 variants. C57BL/6 and CC016J mouse lines were immunized with GI.1 capsid and GII.4 Sydney capsid mRNA in a lipid nanoparticle and sera tested for nAb to GI.1 (FIG. 7A) or GII.4 Sydney (FIG. 7B) in a surrogate assay. At week 32, animals were boosted GII.3/GII.4 P7 chimeric VLP adjuvanted with alhydrogel and a final blood sample collected at week 35. Neutralizing antibody titers to the mRNA vaccine components, antigenically diverse GII.4 pandemic variants, the GII.3/GII.4 chimera, and the GII.3 backbone were then compared at weeks 30 and 35 for C57BL/6 (FIG. 7C) and CC016J (FIG. 7D). ID₅₀, Inhibitory Dose 50%. GMT, Geometric mean titer of nAb before (circles) chimeric GII VLP immunization and three weeks post immunization (squares). Marker, GMT. Error bars, 95% CI. Dashed line, limit of detection. **, P=0.008. *, P<0.03 Wilcoxon test.

FIG. 8 shows a representation of the GII.3/GII.4 “P8” capsid surface. GII.3 dimer (light grey) with GII.3/GII.4 P6 residues (middle grey) and surface exposed residues (dark grey) surrounding broadly neutralizing antibody binding sites conserved across GII.4 variants and predicted to comprise or support enhancement of antibody responses with cross-variant neutralization potency post-vaccination. Epitope and support residues are discontinuous in the genome sequence, but surround known conserved neutralizing antibody epitopes. In some embodiments, epitope residues and support residues may be either conserved between GII.3 and GII.4 or not conserved.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” when used in a claim of this invention is not intended to be interpreted to be equivalent to “comprising.”

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

As used herein, the term “antigen” refers to a molecule capable of inducing the production of immunoglobulins (e.g., antibodies). As used herein, the term “immunogen” refers to when a molecule is capable of inducing a multi-faceted humoral and/or cellular-mediated immune response. In some embodiments, an antigen may be referred to as an immunogen, e.g., under conditions when the antigen is capable of inducing a multi-faceted humoral and/or cellular-mediated immune response. A molecule and/or composition (e.g., including but not limited to a nucleic acid, protein, polysaccharide, ribonucleoprotein (RNP), whole bacterium, and/or composition comprising the same) that is capable of antibody may be referred to as “antigenic” and/or that is capable of immune response stimulation may be referred to as “immunogenic,” and can be said to have the ability of antigenicity and/or immunogenicity, respectively. The binding site for an antibody within an antigen and/or immunogen may be referred to as an epitope (e.g., an antigenic epitope). The term “vaccine antigen,” “vaccine immunogen” or a composition comprising the same (e.g., an immunogenic composition, e.g., a subunit vaccine, e.g., a whole cell vaccine) as used herein refers to such an antigen and/or immunogen as used in a vaccine, e.g., a prophylactic, preventative, and/or therapeutic vaccine. In particular embodiments, an immunogen or antigen can induce a protective immune response against the effects of norovirus infection.

“Effective amount” as used herein refers to an amount of a vector, nucleic acid molecule, epitope, polypeptide, cell, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

The terms “vaccine,” “vaccination,” “vaccinating,” immunizing” and “immunization” are well-understood in the art, and are used interchangeably herein. For example, the terms vaccine, vaccination or immunization can be understood to be a process or composition that produces an immune response to an antigen introduced into the subject via vaccination or immunization of the subject and/or increases a subject's immune reaction or response to an immunogen (e.g., by providing an active immune response), and therefore its ability to resist, overcome and/or recover from infection (i.e., a protective immune response).

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the terms “prime boost immunization,” “prime boost administration,” or “prime and booster” refer to an administration (e.g., immunization) regimen that comprises administering to a subject a primary/initial (priming) administration (e.g., of one or more chimeric norovirus capsid protein of the present invention) and at least one secondary (boosting) administration. In some embodiments, the priming administration and the at least one boosting administration may comprise the same composition, administered in multiple (one or more) repetitions. In some embodiments, the priming administration and the at least one boosting administration may comprise different types of compositions, such as different types of chimeric norovirus capsid proteins of the present invention.

As used herein, the terms “prime immunization,” “priming immunization,” “primary immunization” or “prime” refer to primary antigen stimulation by using a chimeric norovirus capsid protein according to the instant invention.

The term “boost immunization,” “boosting immunization,” “secondary immunization”, or “boost” refers to additional administration (e.g., immunization) of a chimeric norovirus capsid protein of the present invention administered to a subject after a primary administration. In some embodiments, the boost immunization may be administered at a dose higher than, lower than, and/or equal to the dose administered as a primary immunization, e.g., when the boost immunization is administered alone without priming.

The prime and boost vaccine compositions may be administered via the same route or they may be administered via different routes. The boost vaccine composition may be administered one or several times at the same or different dosages. It is within the ability of one of ordinary skill in the art to optimize prime-boost combinations, including optimization of the timing and dose of vaccine administration.

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The terms “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing norovirus infection by the methods of the present invention can be determined by detecting a clinical improvement as indicated by a change in the subject's symptoms and/or clinical parameters (e.g., viremia), as would be well known to one of skill in the art.

Unless indicated otherwise, the terms “protect,” “protecting,” “protection” and “protective” (and grammatical variations thereof) encompass both methods of preventing and treating norovirus infection in a subject, whether against one or multiple strains, genotypes or genogroups of norovirus.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “subject” of the invention includes any animal susceptible to norovirus infection. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. In some embodiments, a laboratory animal may include but is not limited to any standard laboratory mouse strain. In some embodiments, a laboratory animal may include Collaborative cross mouse strains as publicly available through csbio.unc.edu/CCstatus). Subjects of the invention can be a subject known or believed to be at risk of infection by norovirus. Alternatively, a subject according to the invention can also include a subject not previously known or suspected to be infected by norovirus or in need of treatment for norovirus infection.

A “subject in need” of the methods of the invention can be a subject known to be, or suspected of being, infected with, or at risk of being infected with, norovirus.

The present invention is based on the unexpected discovery that epitope regions that define a norovirus capsid protein epitope can be transferred into a protein backbone of a different norovirus genotype to create a chimeric molecule that contains antibody targets for both genotypes, thereby functioning as a bivalent vaccine that can induce neutralizing antibodies against two different norovirus genotypes from a single source. Thus, in one embodiment, the present invention provides a platform for construction of a chimeric norovirus protein capsid backbone that comprises amino acid substitutions that introduce whole or partial epitopes that are recognized by an antibody that is reactive with a norovirus genotype that is different from the norovirus genotype of the norovirus capsid protein backbone.

The amino acid substitutions described herein may not necessarily describe an entire epitope, and the epitope within which the substitutions occur may comprise additional amino acid residues. The inventors of the present invention have characterized multiple epitope regions of the norovirus VP1 capsid protein, as described herein, for example, in WO 2019/079594, the disclosure of which is incorporated herein by reference in its entirety.

For example, in some embodiments, identified residue positions of Epitope I comprise amino acid positions 410, 441, 512, 513, and 514, wherein the numbering is based on the reference amino acid sequence of a norovirus strain GII.3 identified as SEQ ID NO:1. In some embodiments, identified residue positions of the NERK epitope comprise amino acid positions 234, 324, 330, 492, and 501, wherein the numbering is based on the reference amino acid sequence of a norovirus strain GII.3 identified as SEQ ID NO:1. In some embodiments, identified residue positions of epitope F comprise amino acid positions 327 and 412, wherein the numbering is based on the reference amino acid sequence of a norovirus strain GII.3 identified as SEQ ID NO:1. Certain residues, e.g., amino acid positions 492 and/or 501 in the NERK epitope, may be in the same residue in other GII.3 and/or GII.4 strains, but may not necessarily be in the same residue in other GII strains. A different backbone may need a different subset of epitope residues and/or positions substituted to reconstitute particular epitopes, e.g., Epitope I, F, and/or NERK. Epitopes as described herein may comprise additional amino acid residues and/or positions as now known or may later be discovered.

Thus, in some embodiments, the present invention provides a chimeric norovirus (NoV) capsid protein comprising one or more (e.g., 1, 2, 3, 4, 5, 6, 7, 8, 9, 10, or more) amino acid substitutions in any combination that introduce epitopes that are recognized by an antibody that is reactive with a norovirus genotype that is different from the norovirus genotype of the norovirus capsid protein backbone.

In some embodiments, the norovirus capsid protein backbone may be from norovirus genogroup 1 (GI), genogroup 2 (GII) genogroup 3 (GIII), genogroup 4 (GIV), genogroup 5 (GV), genogroup 6 (GVI), and/or genogroup 7 (GVII), including any sub groupings, genotypes, strains, and/or isolates thereof. In some embodiments, the norovirus capsid protein backbone may be from norovirus genogroup 2, genotype 2 (GII.2). In some embodiments, the norovirus capsid protein backbone may be from norovirus genogroup 2, genotype 3 (GII.3). In some embodiments, the norovirus capsid protein backbone may be from norovirus genogroup 2, genotype 4 (GII.4).

It would be understood that any combination of a first norovirus genotype for the norovirus capsid protein backbone and a second norovirus genotype that is the target of the antibody that recognizes the epitope introduced into the norovirus capsid protein backbone can be used, provided that the first norovirus genotype and the second norovirus genotype are different (i.e., not the same genotype). As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

The term “chimeric norovirus capsid protein” and similar terms will be understood in the art to mean a norovirus capsid protein derived from a particular norovirus strain that contains single or multiple amino acid substitutions at various positions in which the amino acid substitution(s) is an amino acid(s) that is one from the corresponding position(s) of a norovirus capsid protein from a different norovirus strain. In representative embodiments, the amino acid substitution comprises residues from a particular epitope from a norovirus strain different from that of the capsid protein in which the substitution is made.

In some embodiments, the amino acid substitutions(s) may be at amino acid residues R341V and/or S412V (Epitope F) of the norovirus VP1 major capsid protein encoded by ORF2 of human norovirus (hNoV), wherein the numbering is based on the reference amino acid sequence of a norovirus strain GII.3 identified as SEQ ID NO:1. In some embodiments, the substitutions may be at amino acid residues amino acid residues D512Q, S513H, and/or P514D (Epitope I) of the norovirus VP1 major capsid protein encoded by ORF2 of human norovirus (hNoV), wherein the numbering is based on the reference amino acid sequence of a norovirus strain GII.3 identified as SEQ ID NO:1. In some embodiments, the amino acid substitution(s) may be at amino acid residues I234V, P324N, and/or D330E (NERK) of the norovirus VP1 major capsid protein encoded by ORF2 of human norovirus (hNoV), wherein the numbering is based on the reference amino acid sequence of a norovirus strain GII.3 identified as SEQ ID NO:1. In some embodiments, the amino acid substitution(s) may be at amino acid residues T254S, E255G, N256A, and/or I257F of the norovirus VP1 major capsid protein encoded by ORF2 of human norovirus (hNoV), wherein the numbering is based on the reference amino acid sequence of a norovirus strain GII.3 identified as SEQ ID NO:1. In some embodiments, the amino acid substitution(s) may be at amino acid residues F408P, K509H, and/or N510T of the norovirus VP1 major capsid protein encoded by ORF2 of human norovirus (hNoV), wherein the numbering is based on the reference amino acid sequence of a norovirus strain GII.3 identified as SEQ ID NO:1. In still further embodiments, the amino acid substitutions may comprise any two or more (e.g., 3, 4, 5, 6, 7, 8, 9, 10, etc.) of these amino acid substitutions and/or epitopes, in any combination.

The term “norovirus capsid protein backbone” and similar terms refer to the particular norovirus capsid protein from which a chimeric norovirus capsid protein is based. The norovirus capsid protein backbone may be from any genogroup, genotype and/or strain of hNoV. In some embodiments of the invention, the norovirus capsid protein backbone is from genogroup II and genotype 3 (GII.3) of hNoV. Other genotypes include GII.1, GII.2, GII.5, GII.6, GII.7 GII.8, GII.9, GII.10, GII.11, GII.12, GII.13, GII.14, GII.15, GII.16, GII.17, GII.18, GII.19, GII.20, GII.21, GII.22, GII.23, GII.24, GII.25, GII.26, as well as any norovirus genotypes now known or later discovered. Non-limiting examples of GII.3 strains include GenBank Access. No. JQ743333 (e.g., SEQ ID NO:1). Non-limiting examples of GII.2 strains include GenBank Access. No. AX065295.1.

In some embodiments, the amino acid substitutions may comprise amino acid substitutions from epitopes of genogroup II and genotype 4 (GII.4). Non-limiting examples of GII.4 strains include GII.4-1974 (GenBank Access. No. ACT76139.1), GII.4-1987 (GenBank Access. No. AAK50355.1), GII.4-1997 (GenBank Access. No. AFJ04707.1), GII.4-2002 (GenBank Access. No. AFJ04708.1), GII.4-2004 (GenBank Access. No. AAZ31376.2), GII.4-2005 (GenBank Access. No. BAE98194.1), GII.4-2006 (GenBank Access. No. AFJ04709.1), GII.4-2007 (GenBank Access. No. BAH56690.1), GII.4-2008a (GenBank Access. No. ACX31885.1), GII.4-2008s (GenBank Access. No. BAH30707.1), GII.4-2009 (GenBank Access. No. ADD10375.1, GII.4-2012 (GenBank Access. No. AFV08795.1), and/or GII.4-2015 (GenBank Access. No. KX907727.1).

In some embodiments, the present invention provides a chimeric norovirus capsid protein comprising one or more (e.g., 1, 2, 3, 4, 5, 6, 7, 8, 9, 10, or more) of the following amino acid substitutions in any combination, wherein the numbering is based on the reference amino acid sequence of a norovirus capsid protein of a norovirus strain GII.3 identified as SEQ ID NO:1: R341V, S412V, D512Q, S513H, P514D, I234V, P324N, and/or D330E.

In some embodiments, the numbering is based on the reference amino acid sequence of a norovirus capsid protein of a norovirus strain GII.3 identified as SEQ ID NO:1.

| GII.3 (GenBank Access. No. JQ743333) | |
|---|---|
| SEQ ID NO: 1 | |
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTISEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTPYDPAED IPAPLGTPDF RGKVFGVASQ RNPDSTTRAH | 360 |
| EAKVDTTSGR FTPKLGSLEI TTESDDFDTN QSTKFTPVGI GVDNEAEFQQ WSLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GDSPITVPPN GYFRFESWVN PFYTLAPMGT | 540 |
| GNGRRRIQ | 549 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); and/or c) amino acid residues I234V, P324N, and D330E (NERK).

In some embodiments, a chimeric norovirus capsid protein of the present invention may further comprise one or more (1, 2, 3, 4, 5, 6, 7, 8, 9, 10, or more) of the following amino acid substitutions in any combination, wherein the numbering is based on the reference amino acid sequence of a norovirus capsid protein of a norovirus strain GII.3 identified as SEQ ID NO:1: T254S, E255G, N256A, I257F, F408P, K509H, and/or N510T.

In some embodiments, the present invention provides a chimeric norovirus capsid protein comprising one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues R341V and S412V (Epitope F).

In some embodiments, a chimeric norovirus capsid protein of this invention can comprise, consist essentially of or consist of the amino acid sequence:

| SEQ ID NO: 2 GII.3.GII.4F | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTISEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTPYDPAED IPAPLGTPDF VGKVFGVASQ RNPDSTTRAH | 360 |
| EAKVDTTSGR FTPKLGSLEI TTESDDFDTN QSTKFTPVGI GVDNEAEFQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GDSPITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues D512Q, S513H, and P514D (Epitope I).

In some embodiments, a chimeric norovirus capsid protein of this invention can comprise, consist essentially of or consist of the amino acid sequence:

| SEQ ID NO: 3 GII.3/GII.4IX | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTISEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTPYDPAED IPAPLGTPDF RGKVFGVASQ RNPDSTTRAH | 360 |
| EAKVDTTSGR FTPKLGSLEI TTESDDFDTN QSTKFTPVGI GVDNEAEFQQ WSLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GQHDITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues R341V and S412V (Epitope F); and amino acid residues D512Q, S513H, and P514D (Epitope I).

In some embodiments, a chimeric norovirus capsid protein of this invention can comprise, consist essentially of or consist of the amino acid sequence:

| SEQ ID NO: 4 GII.3/GII.4F + IX | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTISEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTPYDPAED IPAPLGTPDF VGKVFGVASQ RNPDSTTRAH | 360 |
| EAKVDTTSGR FTPKLGSLEI TTESDDFDTN QSTKFTPVGI GVDNEAEFQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GQHDITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues I234V, P324N, and D330E (NERK).

In some embodiments, a chimeric norovirus capsid protein of this invention can comprise, consist essentially of or consist of the amino acid sequence:

| SEQ ID NO: 5 GII.3/GII.4NERK | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTVSEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTNYDPAEE IPAPLGTPDF RGKVFGVASQ RNPDSTTRAH | 360 |
| EAKVDTTSGR FTPKLGSLEI TTESDDFDTN QSTKFTPVGI GVDNEAEFQQ WSLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GDSPITVPPN GYFRFESWVN PFYTLAPMGT | 540 |
| GNGRRRIQ | 549 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues R341V and S412V (Epitope F); and amino acid residues I234V, P324N, and D330E (NERK).

In some embodiments, a chimeric norovirus capsid protein of this invention can comprise, consist essentially of or consist of the amino acid sequence:

| SEQ ID NO: 6 GII.3/GII.4NERK + F | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTVSEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTNYDPAEE IPAPLGTPDF VGKVFGVASQ RNPDSTTRAH | 360 |
| EAKVDTTSGR FTPKLGSLEI TTESDDFDTN QSTKFTPVGI GVDNEAEFQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GDSPITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues D512Q, S513H, and P514D (Epitope I); and amino acid residues I234V, P324N, and D330E (NERK).

In some embodiments, a chimeric norovirus capsid protein of this invention can comprise, consist essentially of or consist of the amino acid sequence:

| SEQ ID NO: 7 GII.3/GII.4NERK + I | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTVSEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTNYDPAEE IPAPLGTPDF RGKVFGVASQ RNPDSTTRAH | 360 |
| EAKVDTTSGR FTPKLGSLEI TTESDDFDTN QSTKFTPVGI GVDNEAEFQQ WSLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GQHDITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); and c) amino acid residues I234V, P324N, and D330E (NERK).

In some embodiments, a chimeric norovirus capsid protein of this invention can comprise, consist essentially of or consist of the amino acid sequence:

| SEQ ID NO: 8 GII.3/GII.4NERK+F+1 | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTVSEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTNYDPAEE IPAPLGTPDF VGKVFGVASQ RNPDSTTRAH | 360 |
| EAKVDTTSGR FTPKLGSLEI TTESDDFDTN QSTKFTPVGI GVDNEAEFQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GQHDITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: amino acid residues R341V and S412V (Epitope F); and amino acid residues T254S, E255G, N256A, I257F.

In some embodiments, a chimeric norovirus capsid protein of this invention can comprise, consist essentially of or consist of the amino acid sequence:

| SEQ ID NO: 9 GII.3/GII.4 P2 | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTISEMSNS | 240 |
| RFPVPIDSLH TSPSGAFVVQ CQNGRVTLDG ELMGTTQLLP SQICAFRGTL TRSTSRASDQ | 300 |
| ADTPTPRLFN HRWHIQLDNL NGTPYDPAED IPAPLGTPDF VGKVFGVASQ RNPDSTTRAH | 360 |
| EAKVDTTSGR FTPKLGSLEI TTESDDFDTN QSTKFTPVGI GVDNEAEFQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAKN GDSPITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); c) amino acid residues I234V, P324N, and D330E (NERK); and d) amino acid residues F408P, K509H, and/or N510T.

In some embodiments, a chimeric norovirus capsid protein of this invention can comprise, consist essentially of or consist of the amino acid sequence:

| SEQ ID NO: 10 GII.3/GII.4 P3 | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTVSEMSNS | 240 |
| RFPVPIDSLH TSPTENIVVQ CQNGRVTLDG ELMGTTQLLP SQICAFRGTL TRSTSRASDQ | 300 |
| ADTPTPRLFN HRWHIQLDNL NGTNYDPAEE IPAPLGTPDF VGKVEGVASQ RNPDSTTRAH | 360 |
| EAKVDTTSGR FTPKLGSLEI TTESDDFDTN QSTKFTPVGI GVDNEAEPQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAHT GQHDITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); c) amino acid residues I234V, P324N, and D330E (NERK); and d) amino acid residues T254S, E255G, N256A, I257F, F408P, K509H, and N510T.

In some embodiments, a chimeric norovirus capsid protein of this invention can comprise, consist essentially of or consist of the amino acid sequence:

| SEQ ID NO: 11 GII.3/GII.4 P4 | |
|---|---|
| MKMASNDAAP SNDGAAGLVP EINNEAMALD PVAGAAIAAP LTGQQNIIDP WIMNNFVQAP | 60 |
| GGEFTVSPRN SPGEVLLNLE LGPEINPYLA HLARMYNGYA GGFEVQVVLA GNAFTAGKVI | 120 |
| FAAIPPNFPI DNLSAAQITM CPHVIVDVRQ LEPINLPMPD VRNTFFHYNQ DSDSRLRLIA | 180 |
| MLYTPLRANN SGDDVFTVSC RVLTRPSPDF SFNFLVPPTV ESKTKLFTLP ILTVSEMSNS | 240 |
| RFPVPIDSLH TSPSGAFVVQ CQNGRVTLDG ELMGTTQLLP SQICAFRGTL TRSTSRASDQ | 300 |
| ADTPTPRLFN HRWHIQLDNL NGTNYDPAEE IPAPLGTPDF VGKVFGVASQ RNPDSTTRAH | 360 |
| EAKVDTTSGR FTPKLGSLEI TTESDDFDTN QSTKFTPVGI GVDNEAEPQQ WVLPNYSGQF | 420 |
| THNMNLAPAV APNFPGEQLL FFRSQLPSSG GWSNGVLDCL VPQEWVQHFY QESAPAQTQV | 480 |
| ALVRYVNPDT GRVLFEAKLH KLGFMTIAHT GQHDITVPPN GYFRFESWVN PFYTLAPMGT | 540 |
| GNGRRRIQ | 551 |

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); c) amino acid residues I234V, P324N, and D330E (NERK); and d) amino acid residues T254S, E255G, N256A, and I257F.

In some embodiments, a chimeric norovirus capsid protein of this invention can comprise, consist essentially of or consist of the amino acid sequence:

SEQ ID NO: 12 GII.3/GII.4 P5 (F ALL + NERK)
MKMASNDAAPSNDGAAGLVPEINNEAMALDPVAGAAIAAPLTGQQNIIDPWIMNNFVQAPGGEFTVSPRNSPGEV
LLNLELGPEINPYLAHLARMYNGYAGGFEVQVVLAGNAFTAGKVIFAAIPPNFPIDNLSAAQITMCPHVIVDVRQ
LEPINLPMPDVRNTFFHYNQDSDSRLRLIAMLYTPLRANNSGDDVFTVSCRVLTRPSPDESENFLVPPTVESKTK
LFTLPILTVSEMSNSRFPVPIDSLHTSPSGAFVVQCQNGRVTLDGELMGTTQLLPSQICAFRGTLTRSTSRASDQ
ADTPTPRLFNHRWHIQLDNLNGTNYDPAEEIPAPLGTPDFVGKVFGVASQRNPDSTTRAHEAKVDTTSGRFTPKL
GSLEITTESDDFDTNQSTKFTPVGIGVDNEAEFQQWVLPNYSGQFTHNMNLAPAVAPNFPGEQLLFFRSQLPSSG
GWSNGVLDCLVPQEWVQHFYQESAPAQTQVALVRYVNPDTGRVLFEAKLHKLGFMTIAKNGDSPITVPPNGYFRF
ESWVNPFYTLAPMGTGNGRRRIQ

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); c) amino acid residues I234V, P324N, and D330E (NERK); and d) amino acid residues T254S, E255G, N256A, and I257F.

In some embodiments, a chimeric norovirus capsid protein of this invention can comprise, consist essentially of or consist of the amino acid sequence:

SEQ ID NO: 13 GII.3/GII.4 P6 (F ALL + I + NERK)
MKMASNDAAPSNDGAAGLVPEINNEAMALDPVAGAAIAAPLTGQQNIIDPWIMNNFVQAPGGEFTVSPRNSPGEV
LLNLELGPEINPYLAHLARMYNGYAGGFEVQVVLAGNAFTAGKVIFAAIPPNFPIDNLSAAQITMCPHVIVDVRQ
LEPINLPMPDVRNTFFHYNQDSDSRLRLIAMLYTPLRANNSGDDVFTVSCRVLTRPSPDFSFNFLVPPTVESKTK
LFTLPILTVSEMSNSRFPVPIDSLHTSPSGAFVVQCQNGRVTLDGELMGTTQLLPSQICAFRGTLTRSTSRASDQ
ADTPTPRLFNHRWHIQLDNLNGTNYDPAEEIPAPLGTPDFVGKVFGVASQRNPDSTTRAHEAKVDTTSGRFTPKL
GSLEITTESDDFDTNQSTKFTPVGIGVDNEAEFQQWVLPNYSGQFTHNMNLAPAVAPNFPGEQLLFFRSQLPSSG
GWSNGVLDCLVPQEWVQHFYQESAPAQTQVALVRYVNPDTGRVLFEAKLHKLGFMTIAKNGQHDITVPPNGYFRF
ESWVNPFYTLAPMGTGNGRRRIQ

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); and d) amino acid residues T254S, E255G, N256A, and I257F.

In some embodiments, a chimeric norovirus capsid protein of this invention can comprise, consist essentially of or consist of the amino acid sequence:

SEQ ID NO: 14 GII.3/GII.4 P7 (FALL + I)
MKMASNDAAPSNDGAAGLVPEINNEAMALDPVAGAAIAAPLTGQQNIIDPWIMNNFVQAPGGEFTVSPRNSPGEV
LLNLELGPEINPYLAHLARMYNGYAGGFEVQVVLAGNAFTAGKVIFAAIPPNFPIDNLSAAQITMCPHVIVDVRQ
LEPINLPMPDVRNTFFHYNQDSDSRLRLIAMLYTPLRANNSGDDVFTVSCRVLTRPSPDFSFNFLVPPTVESKTK
LFTLPILTISEMSNSRFPVPIDSLHTSPSGAFVVQCQNGRVTLDGELMGTTQLLPSQICAFRGTLTRSTSRASDQ
ADTPTPRLFNHRWHIQLDNLNGTPYDPAEDIPAPLGTPDFVGKVFGVASQRNPDSTTRAHEAKVDTTSGRFTPKL
GSLEITTESDDFDTNQSTKFTPVGIGVDNEAEFQQWVLPNYSGQFTHNMNLAPAVAPNFPGEQLLFFRSQLPSSG
GWSNGVLDCLVPQEWVQHFYQESAPAQTQVALVRYVNPDTGRVLFEAKLHKLGFMTIAKNGQHDITVPPNGYFRF
ESWVNPFYTLAPMGTGNGRRRIQ

In some embodiments, a chimeric norovirus capsid protein of the present invention may comprise one or more (e.g., 1, 2, 3, 4, 5, 6, 7, 8, 9, 10, or more) of the following amino acid substitutions in any combination, wherein the numbering is based on the reference amino acid sequence of a norovirus strain GII.3 identified as SEQ ID NO:1: a) amino acid residues R341V and S412V (Epitope F); b) amino acid residues D512Q, S513H, and P514D (Epitope I); c) amino acid residues I234V, P324N, and D330E (NERK); and d) amino acid residues D247E, S248K, H250F, S252G, T254S, E255G, N256A, I257F, F408P, Q445T, G455M, V456N, K509H, N510T, I515L, T516V, and V517I.

In some embodiments, a chimeric norovirus capsid protein of this invention can comprise, consist essentially of or consist of the amino acid sequence:

SEQ ID NO: 15: GII.3/GII.4 P8
MKMASNDAAPSNDGAAGLVPEINNEAMALDPVAGAAIAAPLTGQQNIIDPWIMNNF
VQAPGGEFTVSPRNSPGEVLLNLELGPEINPYLAHLARMYNGYAGGFEVQVVLAGN
AFTAGKVIFAAIPPNFPIDNLSAAQITMCPHVIVDVRQLEPINLPMPDVRNTFFHYNQD
SDSRLRLIAMLYTPLRANNSGDDVFTVSCRVLTRPSPDFSFNFLVPPTVESKTKLFTLP
ILTVSEMSNSRFPVPIEKVFTGPSGAFVVQCQNGRVTLDGELMGTTQLLPSQICAFR
GTLTRSTSRASDQADTPTPRLFNHRWHIQLDNLNGTNYDPAEEIPAPLGTPDFVGKVF
GVASQRNPDSTTRAHEAKVDTTSGRFTPKLGSLEITTESDDFDTNQSTKFTPVGIGVD
NEAEPQQWVLPNYSGQFTHNMNLAPAVAPNFPGEQLLFFRSTLPSSGGWSNMNLD
CLVPQEWVQHFYQESAPAQTQVALVRYVNPDTGRVLFEAKLHKLGFMTIAHTGQH
DLVIPPNGYFRFESWVNPFYTLAPMGTGNGRRRIQ

In some embodiments, the present invention further provides a synthetic backbone molecule comprising a set of amino acid residues that form a norovirus conformation epitope, comprising the one or more (e.g., 1, 2, 3, 4, or more) of the following set of amino acid substitutions, wherein the numbering is based on the reference amino acid sequence of a norovirus capsid protein of a norovirus strain GII.3 identified as SEQ ID NO:1 a) amino acid residues R341V and S412V (Epitope F); b) amino acid residues D512Q, S513H, and P514D (Epitope I); and/or c) amino acid residues I234V, P324N, and D330E (NERK), wherein the synthetic backbone molecule is not a norovirus capsid protein. In some embodiments, a synthetic backbone molecule of the present invention may further comprise one or more (e.g., 1, 2, 3, 4, 5, 6, 7, or more) of the following amino acid substitutions in any combination: T254S, E255G, N256A, I257F, F408P, K509H, and/or N510T.

In some embodiments, the present invention provides a norovirus P particle (see, Tan et al. (2011) J. Virol. 85(2):753-764) that presents the epitopes of the norovirus capsid proteins as described herein. Thus, in some embodiments, the present invention provides a norovirus P particle comprising a set of amino acid residues that form a norovirus conformation epitope, comprising one or more (e.g., 1, 2, 3, 4, or more) of the following amino acid substitutions in any combination, wherein the numbering is based on the reference amino acid sequence of a norovirus capsid protein of a norovirus strain GII.3 identified as SEQ ID NO:1: a) amino acid residues R341V and S412V (Epitope F); b) amino acid residues D512Q, S513H, and P514D (Epitope I); and/or c) amino acid residues I234V, P324N, and D330E (NERK). In some embodiments, a norovirus P particle of the present invention may further comprise one or more (e.g., 1, 2, 3, 4, 5, 6, 7, or more) of the following amino acid substitutions in any combination: T254S, E255G, N256A, I257F, F408P, K509H, and/or N510T. In some embodiments, the synthetic backbone molecule is not a norovirus capsid protein.

In some embodiments, the present invention provides peptide mimitopes (see, Meloen et al. (2000) J. Mol. Recognit. 13:352-359) that mimic the individual and conformational epitopes of the norovirus capsid proteins of the invention. Mimitopes may be identified using any technique known in the art, such as by surface stimulation, random peptide libraries or phage display libraries, using an antibody or antibodies to the individual and conformational epitopes of the chimeric norovirus capsid proteins of the invention. Thus, in some embodiments, the present invention provides a mimitope comprising a norovirus epitope comprising one or more (e.g., 1, 2, 3, 4, or more) of the following amino acid substitutions in any combination, wherein the numbering is based on the reference amino acid sequence of a norovirus capsid protein of a norovirus strain GII.3 identified as SEQ ID NO:1: a) amino acid residues R341V and S412V (Epitope F); b) amino acid residues D512Q, S513H, and P514D (Epitope I); and/or c) amino acid residues I234V, P324N, and D330E (NERK). In some embodiments, a mimitope of the present invention may further comprise one or more (e.g., 1, 2, 3, 4, 5, 6, 7, or more) of the following amino acid substitutions in any combination: T254S, E255G, N256A, I257F, F408P, K509H, and/or N510T.

In some embodiments, the present invention provides a synthetic nanoparticle and/or scaffold immunogen comprising amino acid residues from about 224 (e.g., from about 210, 211, 212, 213, 214, 215, 216, 217, 218, 219, 220, 221, 223, 224, 225, 226, 227, 228, 229, 230, 231, 232, 233, or 234 at the N terminus of the capsid protein segment) through about 529 (e.g., through about 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, etc. at the C terminus of the norovirus capsid protein segment) of a norovirus capsid protein (e.g., a chimeric norovirus capsid protein), wherein the numbering is based on the reference amino acid sequence of a norovirus capsid protein of a norovirus strain GII.3 identified as SEQ ID NO:1. In some embodiments, the norovirus capsid protein is a segment and not the full norovirus capsid protein. In some embodiments, the immunogen is monovalent. In embodiments, the immunogen is multivalent for different norovirus genogroups, genotypes, strains, and/or isolates.

The present invention further provides a nucleic acid molecule (e.g., an isolated nucleic acid molecule; e.g., an mRNA molecule) encoding the chimeric norovirus capsid protein of this invention, a polypeptide of the invention, a chimeric norovirus Venezuelan Equine Encephalitis (VEE) virus replicon particle (VRP) of the invention and/or a viral coat of the chimeric norovirus particle of the invention.

In some embodiments, the present invention provides a VRP comprising the nucleic acid molecule of this invention. In some embodiments, the present invention provides a virus like particle (VLP) comprising the chimeric norovirus capsid protein of this invention. In an aspect of the chimeric VLP/VRP/virus vaccine approach of the invention, one or more of the identified potential neutralization epitopes from one or more donor strains is moved into any other GII (e.g., GII.2, GII.3, GII.4, etc.) norovirus backbone strain to induce broad protection against multiple strains.

Also provided are vectors encoding the nucleic acid molecules of the invention.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

Accordingly, a vector can be any suitable means for delivering a polynucleotide to a cell. A vector of this invention can be an expression vector that contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a chimeric norovirus capsid protein of this invention is produced in the cell (e.g., a host cell). In addition, the vector of this invention can be in a cell, which can be a cell expressing the nucleic acid of the vector whereby a chimeric norovirus capsid protein of this invention is produced in the cell. It is also contemplated that the nucleic acid molecules and/or vectors of this invention can be present in a host organism (e.g., a transgenic organism), which expresses the nucleic acids of this invention and produces a chimeric norovirus capsid protein of this invention. In some embodiments, the vector is a plasmid, a viral vector, a bacterial vector, an expression cassette, a transformed cell, or a nanoparticle. For example, in some embodiments a chimeric norovirus capsid protein of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

In some embodiments, a nanoparticle vector of the present invention may be an mRNA lipid nanoparticle (mRNA-LNP), a nucleic acid vaccine (NAV), or other nucleic acid lipid nanoparticle compositions, such as described in U.S. Pat. Nos. 9,868,692; 9,950,065; 10,041,091; 10,576,146; 10,702,600; WO2015/164674; US2019/0351048; US2020/297634; WO2020/097548; and Buschmann et al. 2021 Vaccines 9(65) doi.org/10.3390/vaccines9010065; Laczkó et al. 2020 Immunity 53:724-732; and Pardi et al. 2018 Nat. Rev. Drug Discov. 17:261-279, the disclosures of each of which are incorporated herein by reference in their entireties.

Also provided are cells that comprise the vectors, nucleic acid molecules, mimitopes, polypeptides, chimeric norovirus VLPs, chimeric norovirus VRPs and/or chimeric norovirus particles of the invention.

The present invention also provides a composition comprising a chimeric norovirus capsid protein of this invention, a synthetic backbone molecule of this invention, a P particle of this invention, a mimitope of this invention, a synthetic nanoparticle and/or scaffold immunogen of this invention, a nucleic acid molecule of this invention, a vector of this invention, a VRP of this invention and/or a VLP of this invention in a pharmaceutically acceptable carrier. In embodiments, the immunogenic composition is monovalent. In embodiments, the immunogenic composition is multivalent for different norovirus genogroups, genotypes, strains, and/or isolates.

In some embodiments of the invention, the individual and conformational epitopes of the norovirus capsid proteins can be presented on a synthetic backbone or support structure so that the epitopes within the synthetic backbone or support structure mimic the conformation and arrangement of the epitopes within the structure of the norovirus capsid protein, VLP or VRP.

In some embodiments, in order to create a chimeric norovirus construct, the full-length ORF2 major capsid gene sequence from norovirus may be either cloned from a patient sample or produced as a synthetic construct (e.g., from a commercial source). Natural or engineered endonuclease sites may be used to insert sequence containing the desired epitope changes for one or more GII (e.g., GII.2, GII.3, GII.4, etc.) strains. Alternatively, in yet another aspect of the invention, the full-length capsid may be synthesized (e.g., using a consensus sequence) with the desired sequence changes already present. After production of the desired full-length chimeric GII.3 and/or GII.4 norovirus capsid gene, this gene may then be cloned into an expression vector. Upon expression, the VP1 major capsid protein self-assembles into VLPs, which can then be purified. Alternatively, VRPs expressing the major capsid protein may be produced and purified and subsequently used as a vaccine or used as a source of VLP production.

The invention also encompasses methods of vaccinating or immunizing a subject, e.g., to produce an immune response in the subject to norovirus, the method comprising administering to the subject an effective amount of a chimeric norovirus capsid protein, a synthetic backbone molecule, a norovirus P particle, a synthetic nanoparticle and/or scaffold immunogen, a mimitope, a nucleic acid molecule, a polypeptide, a VRP, a vector, a VLP, and/or the composition of the invention.

Subjects may be treated for any purpose, such as for eliciting a protective immune response or for eliciting the production of antibodies in that subject, which antibodies can be collected and used for other purposes such as research or diagnostic purposes or for administering to other subjects to produce passive immunity therein, etc.

Subjects include males and/or females of any age, including neonates, juvenile, mature and geriatric subjects. With respect to human subjects, in representative embodiments, the subject can be an infant (e.g., less than about 12 months, 10 months, 9 months, 8 months, 7 months, 6 months, or younger), a toddler (e.g., at least about 12, 18 or 24 months and/or less than about 36, 30 or 24 months), or a child (e.g., at least about 1, 2, 3, 4 or 5 years of age and/or less than about 14, 12, 10, 8, 7, 6, 5, or 4 years of age). In embodiments of the invention, the subject is a human subject that is from about 0 to 3, 4, 5, 6, 9, 12, 15, 18, 24, 30, 36, 48 or 60 months of age, from about 3 to 6, 9, 12, 15, 18, 24, 30, 36, 48 or 60 months of age, from about 6 to 9, 12, 15, 18, 24, 30, 36, 48 or 60 months of age, from about 9 to 12, 15, 18, 24, 30, 36, 48 or 60 months of age, from about 12 to 18, 24, 36, 48 or 60 months of age, from about 18 to 24, 30, 36, 48 or 60 months of age, or from about 24 to 30, 36, 48 or 60 months of age. In some embodiments, the subject can be an adult, e.g., a human adult.

In some embodiments, the present invention provides a method of producing an immune response to a norovirus virus in a subject, comprising administering to the subject an effective amount of a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention.

In some embodiments, the present invention provides a method of treating a norovirus virus infection in a subject, comprising administering to the subject an effective amount of a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention.

In some embodiments, the present invention provides a method of preventing a disorder associated with norovirus infection in a subject, comprising administering to the subject an effective amount of a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention.

In some embodiments, the present invention provides a method of reducing the risk of developing a disorder associated with norovirus virus infection in a subject, comprising administering to the subject an effective amount of a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention.

In some embodiments, the present invention provides a method of protecting a subject from the effects of norovirus virus infection, comprising administering to the subject an effective amount of a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention.

In some embodiments, the present invention provides a method of detecting a neutralizing antibody to a norovirus, the method comprising determining whether an antibody binds to a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention, wherein binding by the antibody to the chimeric norovirus capsid protein and/or synthetic backbone and/or P particle and/or synthetic nanoparticle and/or scaffold immunogen indicates that the antibody is a neutralizing antibody to a norovirus.

In some embodiments, the present invention provides a method of identifying a neutralizing antibody to a norovirus, comprising: (a) contacting an antibody with a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention; and (b) determining if the antibody binds to the chimeric norovirus capsid protein and/or synthetic backbone and/or P particle and/or synthetic nanoparticle and/or scaffold immunogen, wherein binding by the antibody to the chimeric norovirus capsid protein and/or synthetic backbone and/or P particle and/or synthetic nanoparticle and/or scaffold immunogen identifies the antibody as a neutralizing antibody to a norovirus.

In some embodiments, the present invention provides a method of identifying an immunogenic composition that induces a neutralizing antibody to a norovirus in a subject, the method comprising: (a) contacting a biological sample from a subject that has been administered the immunogenic composition with a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention; (b) determining if the biological sample comprises an antibody that binds the chimeric norovirus capsid protein and/or synthetic backbone and/or P particle and/or synthetic nanoparticle and/or scaffold immunogen; and (c) identifying the immunogenic composition as inducing a neutralizing antibody to a norovirus in the subject if the biological sample comprises an antibody that binds to the chimeric norovirus capsid protein and/or synthetic backbone and/or P particle and/or synthetic nanoparticle and/or scaffold immunogen.

In some embodiments, the present invention provides a method of identifying an immunogenic composition that induces a neutralizing antibody to a norovirus in a subject, the method comprising: (a) administering an immunogenic composition comprising a norovirus antigen to a subject in an amount effective to induce antibodies against the norovirus antigen; (b) contacting a biological sample from the subject with a chimeric norovirus capsid protein, a synthetic backbone molecule, a norovirus P particle, a synthetic nanoparticle and/or scaffold immunogen, a mimitope, a nucleic acid molecule, a polypeptide, a cell, a VRP, a vector, a VLP, and/or the composition of the invention; (c) determining if the biological sample comprises an antibody that binds the chimeric norovirus capsid protein and/or synthetic backbone and/or P particle and/or synthetic nanoparticle and/or scaffold immunogen; and (d) identifying the immunogenic composition as inducing a neutralizing antibody to a norovirus in the subject if the biological sample comprises an antibody that binds the chimeric norovirus capsid protein and/or synthetic backbone and/or P particle and/or synthetic nanoparticle and/or scaffold immunogen.

Pharmaceutical formulations (e.g., immunogenic formulation) comprising the norovirus epitopes, chimeric norovirus capsid proteins, polypeptides, chimeric norovirus VLPs, chimeric norovirus VRPs or chimeric norovirus particles, nucleic acids, vectors, cells or compositions of the invention and a pharmaceutically acceptable carrier are also provided, and can be formulated for administration in a pharmaceutical carrier in accordance with known techniques. See, e.g., Remington, The Science and Practice of Pharmacy (latest edition). In the manufacture of a pharmaceutical composition according to embodiments of the present invention, the composition of the invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of the invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients. In certain embodiments, the pharmaceutically acceptable carrier is sterile and would be deemed suitable for administration into human subjects according to regulatory guidelines for pharmaceutical compositions comprising the carrier.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

In some embodiments, the compositions of the invention can further comprise one or more than one adjuvant. The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al., Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

An adjuvant for use with the present invention, such as, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

In embodiments of the invention, the adjuvant comprises an alphavirus adjuvant as described, for example in U.S. Pat. No. 7,862,829.

Boosting dosages can further be administered over a time course of days, weeks, months or years. In chronic infection, initial high doses followed by boosting doses may be advantageous.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science and Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin may take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, e.g., Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

In embodiments of the invention, a dosage of a virus particle of this invention can be in a range of about 10³ to about 10⁸ plaque forming units (PFUs; e.g., about 10³, about 10⁴, about 10⁵, about 10⁶, about 10⁷, or about 10⁸ PFUs or any value or range therein). In embodiments of this invention, a dosage of a VLP of this invention can be in a range of about 5 micrograms to 5 milligrams (e.g., about 5 micrograms, about 100 micrograms, about 750 micrograms, about 1000 micrograms, about 2.5 milligrams, about 5 milligrams, or any value or range therein). In embodiments of this invention, a dosage of a protein of this invention can be in a range of about 10 to about 10⁵ micrograms+/−adjuvant (e.g., about 10 micrograms, about 100 micrograms, about 500 micrograms, about 1000 micrograms, about 10² micrograms, about 10³ micrograms, about 10⁴ micrograms, about 5×10⁴ micrograms, about 10⁵ micrograms, or any value or range therein).

The present subject matter will now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following examples provide illustrative embodiments. Certain aspects of the following examples are disclosed in terms of techniques and procedures found or contemplated by the present inventors to work well in the practice of the embodiments. In light of the present disclosure and the general level of skill in the art, those of skill will appreciate that the following examples are intended to be exemplary only and that numerous changes, modifications, and alterations can be employed without departing from the scope of the presently claimed subject matter.

### Example 1

Regulating domains of human norovirus GII.4 antibody-meditated immunity. The HuNoV plus-sensed RNA genome encodes 3 open reading frames (ORFs): a 1738 amino acid (AA) replicase (ORF1), a major capsid protein of ˜530 AA (ORF2: VP1), and a 212 AA minor capsid protein (ORF3: VP2). Subdivided into six genogroups (GI-GVI), the HuNoV GI and GII strains are ˜60% different and cause ˜5% and 95% of the recent human outbreaks. Although the GI and GII are further subdivided into ˜8 and 24+ genotypes, respectively, that differ by at least ˜30%, HuNoV serogroups are poorly defined. In 2017-18, the most frequent strains were a GII.4-2015 Sydney variant and GII.2 Snow Mountain Virus (SMV). GII.4 causes 50-90% of the outbreaks each year since 1997 and cause the most severe disease with GII.2 outbreaks increasing since 2015. Noroviruses are stable, naked, icosahedral viruses of 38 nM. The 180 VP1 monomers self-assemble into 90 dimers, which self-assemble into virus-like particles (VLPs) that are antigenically indistinguishable from HuNoV virions.

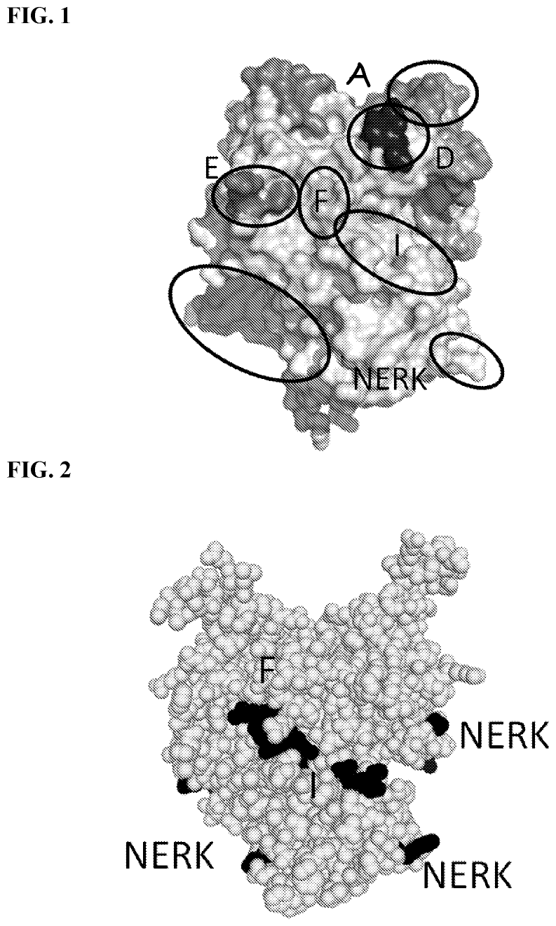

The monomeric VP1 capsid protein is divided into a shell (S) and protruding domain (P), linked by a flexible hinge. The P domain is sub-divided into P1 and P2 and forms prominent protrusions that extend away from the capsid. The P2 (amino acids 279-413; wherein the numbering is based on the reference amino acid sequence of a norovirus capsid protein of a norovirus strain GII.3 identified as SEQ ID NO:1) surface contains the determinants of strain specificity and histoblood group antigens (HBGA) ligand binding, while specific internal residues in the particle core regulate conformational shifts (“breathing core”). The human norovirus capsid surface is comprised of surface protrusions (P domains), formed of dimers of the capsid protein (FIG. 1). P domain motifs interact with host cellular ligands and neutralizing antibodies (Table 1). Antibodies that bind to neutralizing/ligand binding blocking epitopes correlate with protection from infection. Viral evolution in epitopes A, D, and E correlate with immune escape and pandemic strain emergence. Epitopes F, G and I are neutralizing/blocking and conserved across GII.4 pandemic strains. NERK residues are a set of internal “breathing core” residues that regulate presentation of highly conserved epitope F surface residues. Residue modifications may introduce changes into the breathing core sites, designed to open up conserved blockade epitopes that are protected by particle structural dynamics. A vaccine that enhances development of antibodies to epitopes F, G and I may provide breadth of protection to newly emergent GII.4 strains that are divergent in hypervariable epitopes.

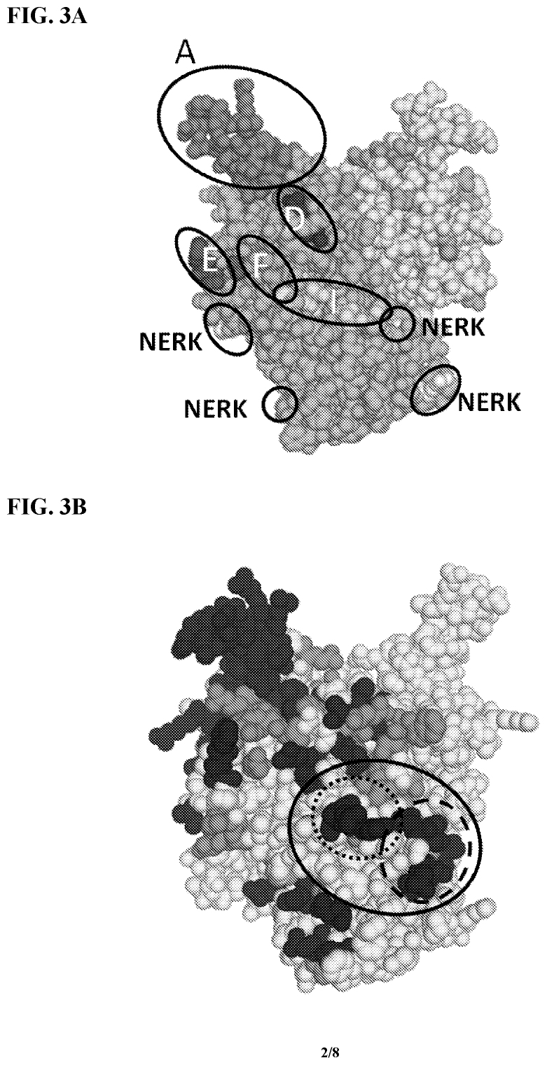

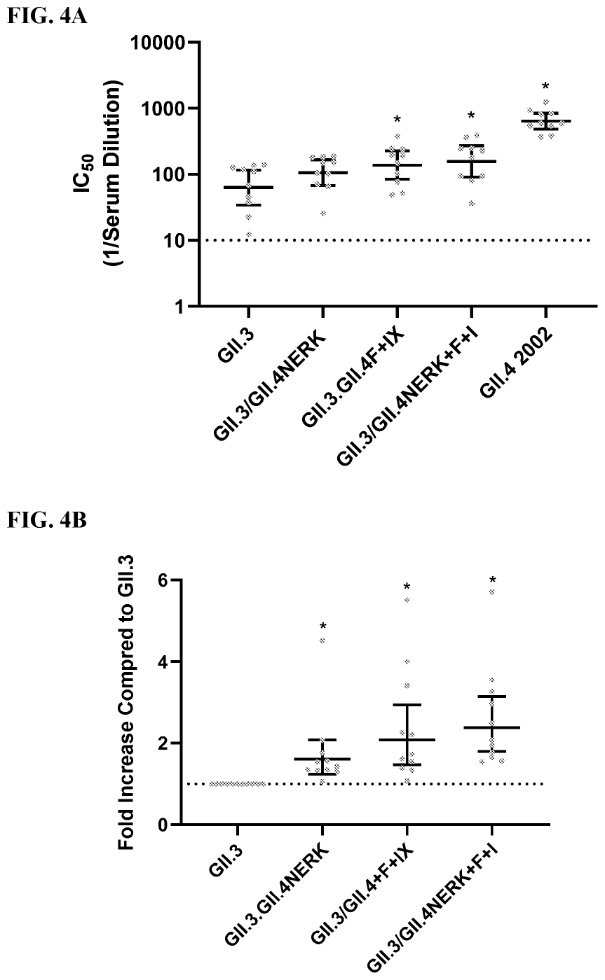

In this study, chimeric genogroup II VLP with GII.4 motifs epitope F, epitope I and NERK were genetically transplanted individually or in combination into the GII.3 backbone creating GII epitope chimeric VLP with the particle conformation breathing core allowing optimum antibody access to the GII.4 conserved epitopes (Table 2). Motifs are highlighted by color and grayscale as in FIG. 1 on a monomer of the GII.3 dimer (FIGS. 2 and 3A). GII strains of human norovirus account for >90% of all infections. GII.3 norovirus is a leading cause of childhood acute gastroenteritis globally. A chimeric GII vaccine candidate with GII.4 conserved epitopes incorporated could provide breadth of cross-GII protection. All chimeric GII.3 VLP were stable.

A subset of GII.3/GII.4 chimeric VLP were evaluated for gain of function of monoclonal antibodies to GII.4 conserved neutralizing/blocking epitopes in a surrogate neutralization assay and the amount of antibody (IC₅₀, μg/ml) needed to block 50% of VLP binding to ligand determined by non-linear fit of sigmoidal curves in GraphPad Prism 8 (Table 3). Boxes with values less than 4 show gain of epitope-specific blockade of ligand binding potency. Genetic implantation of amino acids comprising GII.4 epitopes F and I gained blockade potency for antibodies to GII.4 epitopes F and I. Inclusion of the NERK residues, amino acids outside the antibody binding site, were required for gain of epitope G monoclonal antibody potency and improved epitope F mAb potency. These data support the feasibility of genetic transplantation of key epitopes between GII strains and emphasize the necessity of inclusion of residues outside the antibody-binding site to resurface the antigenic landscape of the GII chimeric VLP for optimum epitope presentation.

Blockade antibody potency for GII.3/GII.4+F+I+NERK is similar to potency for GII.4 strains for antibodies to conserved GII.4 blockade epitopes, as shown in Table 4. GII.3/GII.4+F+I+NERK chimeric VLP and a panel of time-ordered pandemic GII.4 strains were evaluated for gain of neutralization (ligand-blockade) function for monoclonal antibodies to GII.4 conserved neutralizing/blocking epitopes in a surrogate neutralization assay and the amount of antibody (IC₅₀, μg/ml) needed to block 50% of VLP binding to ligand determined by non-linear fit of sigmoidal curves in GraphPad Prism 8.

Undefined residues outside the F, I and NERK motifs mediate monoclonal antibody blockade potency. The amount of antibody needed to block 50% of VLP binding to ligand (IC₅₀, μg/ml) varies across time-ordered pandemic GII.4 strains and GII.3/GII.4 F+I+NERK, indicating that additional, undefined residues modulate antibody potency either by direct antibody binding or allosteric mechanisms of landscape resurfacing. Mapping of GII.4 epitopes onto the monomer of the GII.3 P dimer (FIG. 3A) and comparing the sequence divergences between GII.3 and GII.4 2002 (FIG. 3B; medium grey=conserved changes, black=non-conserved changes), identifies residues surrounding epitope F and I (dashed circled) that may improve presentation of GI.4 conserved neutralizing/blocking epitopes and subsequently boost cross-immunity following chimeric VLP vaccination. From these analyses, two large patches of nearby residues that could influence GII.4 conserved epitope presentation in the context of the GII.3 VLP were identified. Importantly, the same approach can be applied to other GII strains, as dictated by epidemiological surveillance data.

Using sequence variation between GII.3 and GII.4 within the region of interest (FIG. 3B, circled), additional chimeric VLP were designed to further remodel the GII.3 ROI with GII.4 conserved residues (Table 2; FIG. 3B). These additional changes may improve binding of antibody to GII.4 epitope G and enhance serum neutralization potency.

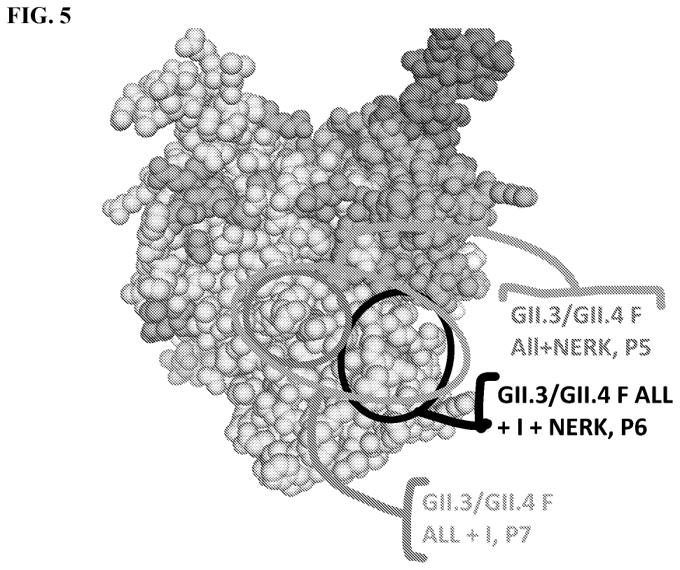

Transplantation of GII.4 conserved neutralizing epitopes into human norovirus GI.3 VLP improves neutralization ligand blocking potency of human sera. Residues comprising GII.4 domains NERK, F+I, or NERK+F+I were genetically transplanted into the GII.3 backbone and tested for neutralization by sera collected from healthy adults. GII.4 antigenic region (F+I and NERK+F+I) transplanted into GII.3 backbone improved the neutralizing potency of serum compared to GII.3 (FIG. 4A). The fold increase in GII.3 neutralizing antibody potency was compared across the GII.3/GII.4 chimeric antigens for each individual. NERK, F+I and NERK+F+I transplantation into GII.3 improved serum blockade potency (FIG. 4B).

These studies were performed transplanting GII.4 conserved epitopes into a non-evolving GII backbone such as GII.3, to design a human norovirus immunogen that provides breadth and durability of immunity. GII.3 viruses cause about 10% of norovirus infections in children but are antigenically stable. Resurfacing local landscape to improve antibody access to conserved occluded epitopes may involve removing local steric hindrance and/or modifying particle formation through allosteric mechanisms.

### Example 2

Additional mapping was performed and additional chimeric VLPs were generated according to the methods as described in Example 1. Table 5 indicates the mapped location of additional residues comprising Epitope F. Transplantation of residues 254-257, 341, and 412 (wherein the numbering corresponds to GII.3 such as reference SEQ ID NO:1) reconstituted GII.4 conserved blockade Epitope F in the GII.3 backbone, conferring antibody blockade potency equivalent to native GII.4 potency in the GII chimeric VLP. Transplantation of these residues did not impact potency of antibodies to epitopes G or I. Combined residues 254-257, 341, and 412 comprise Epitope F All in GII.3 numbering. Residues 254-257, 327, and 404 comprise Epitope F All in GII.4 2002 (GenBank® Accession No. JQ478408) numbering.

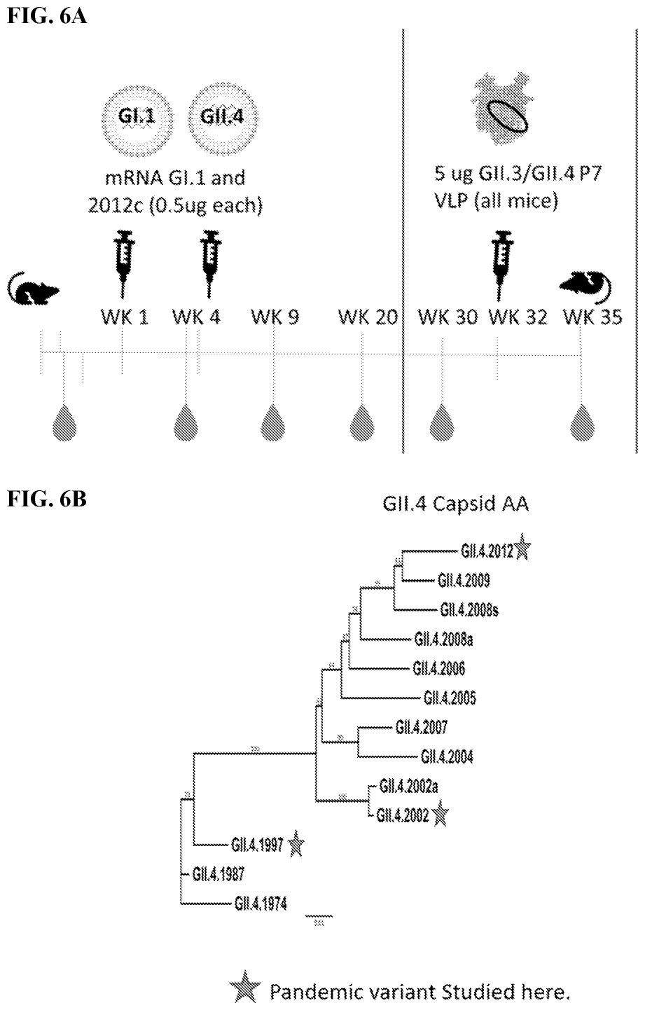

GII.3/GII.4 chimeric VLPs showed improved presentation of conserved GII.4 blockade antibody epitopes. Using sequence variation between GII.3 and GII.4 within the regions of interest (FIG. 5; circled), additional chimeric VLPs were designed to further remodel the GII.3 region of interest with GII.4 conserved residues, including Epitope F All. The NERK motif was included in VLP P5 and P6 (Table 6).

Table 7 shows human monoclonal antibody IC50 values against GII.4 conserved neutralizing antibody epitopes in comparison between parental strains GII.3, GII.4, and chimeric GII.3/GII.4 P7 (GII.3/GII.4 F ALL+I).

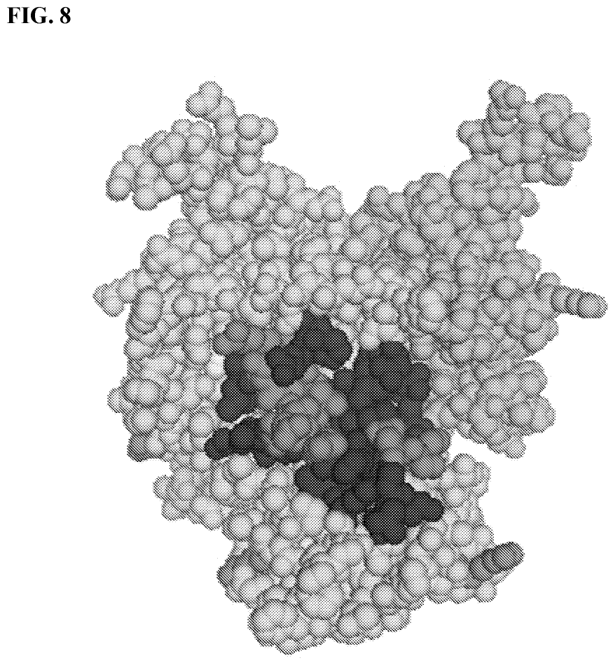

An additional chimeric VLP, P8, was generated comprising the sequence of P6 with additional support residues. The one or more substitutions made in P8 are provided in Table 8. A schematic of P8 capsid surface is shown in FIG. 8. The sequence of GII.3/GII.4 P8 is shown below with P6 and support residues bolded.

SEQ ID NO: 15: GII.3/GII.4 P8
MKMASNDAAPSNDGAAGLVPEINNEAMALDPVAGAAIAAPLTGQQNIIDPWIMNNF
VQAPGGEFTVSPRNSPGEVLLNLELGPEINPYLAHLARMYNGYAGGFEVQVVLAGN
AFTAGKVIFAAIPPNFPIDNLSAAQITMCPHVIVDVRQLEPINLPMPDVRNTFFHYNQD
SDSRLRLIAMLYTPLRANNSGDDVFTVSCRVLTRPSPDFSFNFLVPPTVESKTKLFTLP
ILTVSEMSNSRFPVPIEKVFTGPSGAFVVQCQNGRVTLDGELMGTTQLLPSQICAFR
GTLTRSTSRASDQADTPTPRLFNHRWHIQLDNLNGTNYDPAEEIPAPLGTPDFVGKVF
GVASQRNPDSTTRAHEAKVDTTSGRFTPKLGSLEITTESDDFDTNQSTKFTPVGIGVD
NEAEPQQWVLPNYSGQFTHNMNLAPAVAPNFPGEQLLFFRSTLPSSGGWSNMNLD
CLVPQEWVQHFYQESAPAQTQVALVRYVNPDTGRVLFEAKLHKLGFMTIAHTGQH
DLVIPPNGYFRFESWVNPFYTLAPMGTGNGRRRIQ

### Example 3

Studies were performed that demonstrated GII chimeric VLP booster post mRNA vaccination broadens neutralizing antibody (nAb) responses to the mRNA vaccine components, additional genotypes, and antigenically divergent GII.4 variants.

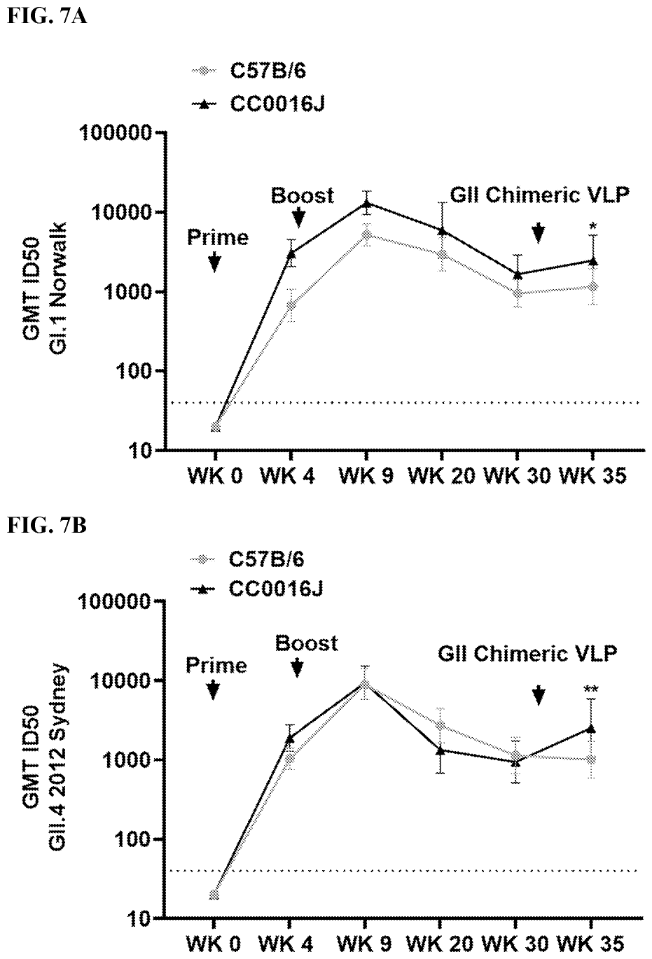

Experiments were performed to model a prime-boost mRNA vaccination regimen (FIG. 6A). In brief, mRNA of GI.1 and GII.4 norovirus strain capsid (VP1) were administered to mice at a dose of 0.5 μg each, with blood samples collected prior to administration as well 5 blood samples analyzed at week 4, 9, 20, 30, and 35. At week 32, 5 μg of GI.3/GII.4.P7 VLP was administered to all mice. A phylogenetic tree of norovirus GII.4 variants, including those tested, is shown in FIG. 6B.

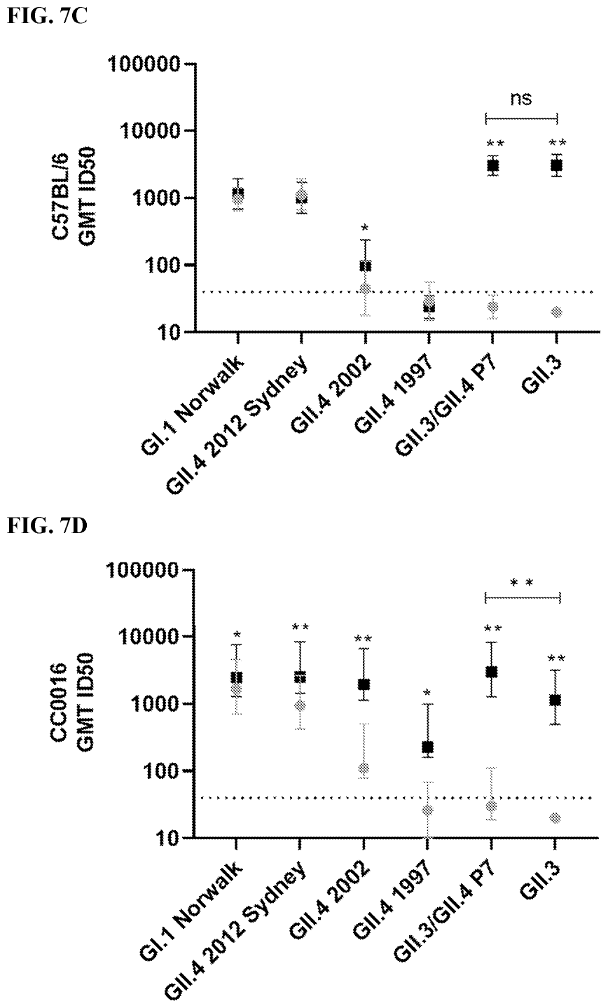

FIGS. 7A-7D show experimental results from C57BL/6 and collaborative cross CC016J mouse lines immunized with equal parts GI.1 capsid and GII.4 Sydney capsid mRNA in a lipid nanoparticle and sera tested for neutralizing antibody (nAb) to GI.1 (FIG. 7A) or GII.4 Sydney (FIG. 7B), as described above. The collaborative cross mouse strains are based on 8 founders, with genome inter-dispersion of parental lines and allow for gene-level mapping of a spectrum of phenotypes, as reviewed in Enriquez et al., 2020 BMC Immunology 21:50, incorporated herein by reference. mRNA vaccination induced robust and durable nAb titer to both vaccine components, Inhibitory Dose 50% (ID50) geometric mean titer (GMT)>900 for at least 24 weeks post-boost for both norovirus genotypes. At week 32, animals were boosted with GII.3/GII.4 P7 chimeric VLP adjuvanted with alhydrogel and a final blood sample collected at week 35. The nAb titers were then compared at weeks 30 and 35 for C57BL/6 (FIG. 7C) and CC016J (FIG. 7D) to evaluate the titer and breadth of nAb generated in response to the GII.3/GII.4 chimera vaccine. C57BL/6 mice responded similarly to the chimeric VLP (GMT 3022) and the GII.3 backbone (GMT 3047). Chimeric VLP vaccination in C57BL/6 mice boosted GII.4 cross-nAb to the 2002 pandemic variant (GMT 98) but not to the more divergent 1997 pandemic variant (GMT 24). Chimeric immunization of CC016J mice with the chimeric VLP induced higher titer of nAb to the chimeric VLP (GMT 2983) than to the GII.3 backbone (GMT 1136). Subsequently, CC016J mice had significant increases in nAb to GII.4 2012 (GMT 2511), GII.4 2002 (GMT 1949) and GII.4 1997 (GMT 228). These data indicate the conserved GII.4 nAb epitopes in the GII.3 backbone were correctly presented and immunogenic in vivo within the chimeric VLP backbone. Antibodies to the conserved epitopes were boosted with chimeric VLP vaccination, supporting GII.3/GII.4 chimeric VLP as candidate vaccine immunogens to generate broad antibody immunity to multiple norovirus genotypes and to antigenic variants of the pandemic GII.4 noroviruses.

While there are shown and described particular embodiments of the invention, it is to be understood that the invention is not limited thereto but may be otherwise variously embodied and practiced within the scope of the following claims. Since numerous modifications and alternative embodiments of the present invention will be readily apparent to those skilled in the art, this description is to be construed as illustrative only and is for the purpose of teaching those skilled in the art the best mode for carrying out the present invention. Accordingly, all suitable modifications and equivalents may be considered to fall within the scope of the following claims.

TABLE 1

| Motif | Features |
|---|---|
| Epitope A | Blockade antibody epitope |
| | Hypervariable |
| | Immunodominant |
| | Exterior surface facing |
| Epitope D | Blockade antibody epitope |
| | Hypervariable |
| | Modulates ligand affinity |
| | Exterior surface facing |
| Epitope E | Blockade antibody epitope |
| | Hypervariable |
| | Antibody access mediated by particle conformation dynamics |
| Epitope F | Blockade antibody epitope |
| | Conserved across GII.4 pandemic strains |
| | Antibody access mediated by particle conformation dynamics |
| Epitope I | Blockade antibody epitope |
| | Conserved across GII.4 pandemic strains |
| | Antibody access mediated by particle conformation dynamics |
| Epitope G | Blockade antibody epitope (recognized by monoclonal antibody 66.694) |
| | Conserved across GII.4 pandemic strains |
| | Antibody access mediated by particle conformation dynamics |
| | Residues undefined |
| NERK | “Breathing Core” allosteric regulation of antibody access to conserved GII.4 blocking |
| | antibody epitopes by mediating particle conformation |

TABLE 2
Chimeric GII VLP with combinations of motifs involved in antibody-mediated immunity.

| | Epitope | | | | | | | | | | | |
|---|---|---|---|---|---|---|---|---|---|---|---|---|
| GII.3 | NERK | | | | | NERK | NERK | NERK | NERK | F | | |
| Residue | 234 | 254 | 255 | 256 | 257 | 324 | 330 | 492 | 501 | 341 | 408 | 409 |
| GII.3 | I | T | E | N | I | P | D | R | K | R | F | Q |
| GII.3/ | I | T | E | N | I | P | D | R | K | V | F | Q |
| GII.4F | | | | | | | | | | | | |
| GII.3/ | I | T | E | N | I | P | D | R | K | R | F | Q |
| GII.4IX | | | | | | | | | | | | |
| GII.3/ | I | T | E | N | I | P | D | R | K | V | F | Q |
| GII.4F + IX | | | | | | | | | | | | |
| GII.3/ | V | T | E | N | I | N | E | R | K | R | F | Q |
| GII.4NERK | | | | | | | | | | | | |
| GII.3/ | V | T | E | N | I | N | E | R | K | V | F | Q |
| GII.4NERK + F | | | | | | | | | | | | |
| GII.3/ | V | T | E | N | I | N | E | R | K | R | F | Q |
| GII.4NERK + I | | | | | | | | | | | | |
| GII.3/ | V | T | E | N | I | N | E | R | K | V | F | Q |
| GII.4NERK + | | | | | | | | | | | | |
| F + I | | | | | | | | | | | | |
| GII.3/ | I | S | G | A | F | P | D | R | K | V | F | Q |
| GII.4 P2 | | | | | | | | | | | | |
| GII.3/ | V | T | E | N | I | N | E | R | K | V | P | Q |
| GII.4 P3 | | | | | | | | | | | | |
| GII.3/ | V | S | G | A | F | N | E | R | K | V | P | Q |
| GII.4 P4 | | | | | | | | | | | | |
| GII.4 | V | S | G | A | F | N | E | R | K | V | P | Q |
| 2002 | | | | | | | | | | | | |

| | Epitope | | | | | | | | | | |
|---|---|---|---|---|---|---|---|---|---|---|---|
| GII.3 | I | I | F | | | | | | I | I | I |
| Residue | 410 | 411 | 412 | 413 | 416 | 509 | 510 | 511 | 512 | 513 | 514 |
| GII.3 | Q | W | S | L | P | K | N | G | D | S | P |
| GII.3/ | Q | W | V | L | P | K | N | G | D | S | P |
| GII.4F | | | | | | | | | | | |
| GII.3/ | Q | W | S | L | P | K | N | G | Q | H | D |
| GII.4IX | | | | | | | | | | | |
| GII.3/ | Q | W | V | L | P | K | N | G | Q | H | D |
| GII.4F + IX | | | | | | | | | | | |
| GII.3/ | Q | W | S | L | P | K | N | G | D | S | P |
| GII.4NERK | | | | | | | | | | | |
| GII.3/ | Q | W | V | L | P | K | N | G | D | S | P |
| GII.4NERK + F | | | | | | | | | | | |
| GII.3/ | Q | W | S | L | P | K | N | G | Q | H | D |
| GII.4NERK + I | | | | | | | | | | | |
| GII.3/ | Q | W | V | L | P | K | N | G | Q | H | D |
| GII.4NERK + | | | | | | | | | | | |
| F + I | | | | | | | | | | | |
| GII.3/ | Q | W | V | L | P | K | N | G | D | S | P |
| GII.4 P2 | | | | | | | | | | | |
| GII.3/ | Q | W | V | L | P | H | T | G | Q | H | D |
| GII.4 P3 | | | | | | | | | | | |
| GII.3/ | Q | W | V | L | P | H | T | G | Q | H | D |
| GII.4 P4 | | | | | | | | | | | |
| GII.4 | Q | W | V | L | P | H | T | G | Q | H | D |
| 2002 | | | | | | | | | | | |

TABLE 3
Monoclonal Antibody Potency, IC₅₀ μg/ml.

| | Epitope | Epitope | Epitope |
|---|---|---|---|
| | F mAb | G mAb | I mAb |
| GII.3 | >4 | >4 | >4 |
| GII.3/GII.4 NERK | >4 | >4 | >4 |
| GII.3/GII.4 + F | 0.41 | >4 | >4 |
| GII.3/GII.4 + F + NERK | 0.55 | >4 | >4 |
| GII.3/GII.4 + I | >4 | >4 | 0.52 |
| GII.3/GII.4 + I + NERK | >4 | 1.6 | 0.43 |
| GII.3/GII.4 + F + I | 0.20 | >4 | 0.13 |
| GII.3/GII.4 + F + I + NERK | 0.07 | 1.8 | 0.13 |

TABLE 4
Monoclonal Antibody Potency, IC₅₀ μg/ml.

| | Epitope F | Epitope G | Epitope I |
|---|---|---|---|
| GII.4 1997 | 0.52 | 3.7 | 1.1 |
| GII.4 2002 | 0.07 | 1.4 | 0.33 |
| GII.4 2006b | 0.61 | 3.2 | 0.39 |
| GII.4 2009 | 0.48 | 2.2 | 1.4 |
| GII.4 2012 | 0.68 | 2.0 | 1.5 |
| GII.3/GII.4 + F + I + NERK | 0.07 | 1.8 | 0.13 |

TABLE 5
Additional residues comprising Epitope F.

| GII.3.GII.4 | Epitope | | | | | | | | | | | |
|---|---|---|---|---|---|---|---|---|---|---|---|---|
| Chimeric | IC50 | IC50 | IC50 | NERK | | | | | NERK | NERK | NERK | NERK |
| VLP | NVB71 | 66.694 | 1431 | 234 | 254 | 255 | 256 | 257 | 324 | 330 | 492 | 501 |
| GII.3 | >4 | >4 | >4 | I | T | E | N | I | P | D | R | K |
| GII.3/GII.4 | 0.07 | >4 | >4 | I | S | G | A | F | P | D | R | K |
| P2 opt F | | | | | | | | | | | | |
| All | | | | | | | | | | | | |
| GII.4 | 0.07 | 1.4 | 0.33 | V | S | G | A | F | N | E | R | K |
| 2002 | | | | | | | | | | | | |

| GII.3.GII.4 | Epitope | | | | | | | | | | | | | |
|---|---|---|---|---|---|---|---|---|---|---|---|---|---|---|
| Chimeric | F | | | I | I | F | | | | | | I | I | I |
| VLP | 341 | 408 | 409 | 410 | 411 | 412 | 413 | 416 | 509 | 510 | 511 | 512 | 513 | 514 |
| GII.3 | R | F | Q | Q | W | S | L | P | K | N | G | D | S | P |
| GII.3/GII.4 | V | F | Q | Q | W | V | L | P | K | N | G | D | S | P |
| P2 opt F | | | | | | | | | | | | | | |
| All | | | | | | | | | | | | | | |
| GII.4 | V | P | Q | Q | W | V | L | P | H | T | G | Q | H | D |
| 2002 | | | | | | | | | | | | | | |

TABLE 6
Additional GII.3/GII.4 chimeric VLPs for improved
presentation of conserved GII.4 blockade antibody.

| | Epitope | | | | | | | | | | |
|---|---|---|---|---|---|---|---|---|---|---|---|
| GII.3 | NERK | | | | | NERK | NERK | NERK | NERK | F | |
| Residue | 234 | 254 | 255 | 256 | 257 | 324 | 330 | 492 | 501 | 341 | 408 |
| GII.3 | I | T | E | N | I | P | D | R | K | R | F |
| GII.3/ | V | S | G | A | F | N | E | R | K | V | F |
| GII.4P | | | | | | | | | | | |
| 5 F ALL + | | | | | | | | | | | |
| NERK | | | | | | | | | | | |
| GII.3/ | V | S | G | A | F | N | E | R | K | V | F |
| GII.4P | | | | | | | | | | | |
| 6 Fall + | | | | | | | | | | | |
| I + NERK | | | | | | | | | | | |
| GII.3/ | I | S | G | A | F | P | D | R | K | V | F |
| GII.4P | | | | | | | | | | | |
| 7 Fall + I | | | | | | | | | | | |
| GII.4 | V | S | G | A | F | N | E | R | K | V | P |
| 2002 | | | | | | | | | | | |

| | Epitope | | | | | | | | | | | |
|---|---|---|---|---|---|---|---|---|---|---|---|---|
| GII.3 | | I | I | F | | | | | | I | I | I |
| Residue | 409 | 410 | 411 | 412 | 413 | 416 | 509 | 510 | 511 | 512 | 513 | 514 |
| GII.3 | Q | Q | W | S | L | P | K | N | G | D | S | P |
| GII.3/ | Q | Q | W | V | L | P | K | N | G | D | S | P |
| GII.4P | | | | | | | | | | | | |
| 5 F ALL + | | | | | | | | | | | | |
| NERK | | | | | | | | | | | | |
| GII.3/ | Q | Q | W | V | L | P | K | N | G | Q | H | D |
| GII.4P | | | | | | | | | | | | |
| 6 Fall + | | | | | | | | | | | | |
| I + NERK | | | | | | | | | | | | |
| GII.3/ | Q | Q | W | V | L | P | K | N | G | Q | H | D |
| GII.4P | | | | | | | | | | | | |
| 7 Fall + I | | | | | | | | | | | | |
| GII.4 | Q | Q | W | V | L | P | H | T | G | Q | H | D |
| 2002 | | | | | | | | | | | | |

TABLE 7
HumAb to GII.4 Conserved nAb Epitopes

| VLP | IC₅₀ Epi F | IC₅₀ Epi I | IC₅₀ Epi X |
|---|---|---|---|
| GII.3 | >4 | >4 | >4 |
| GII.3/GII.4 P7 | 0.01 | 0.06 | 3.7 |
| GII.4 | 0.07 | 0.33 | 1.6 |

TABLE 8
Positions of substitutions and example substitutions
of GII.3/GII.4 P8, wherein the numbering
is based on the reference amino acid sequence of a
norovirus strain GII.3 identified as SEQ ID NO: 1.

| | | Non-Limiting | |
|---|---|---|---|
| Position of | | Examples of | |
| Substitution | | Substitution | |
| 234 | 438 | I234V | Q438Q |
| 246 | 439 | I246I | L439L |
| 247 | 440 | D247E | L440L |
| 248 | 441 | S248K | F441F |
| 249 | 442 | L249L | F442F |
| 250 | 443 | H250F | R443R |
| 251 | 444 | T251T | S444S |
| 252 | 445 | S252G | Q445T |
| 253 | 454 | P253P | N454N |
| 254 | 455 | T254S | G455M |
| 255 | 456 | E255G | V456N |
| 256 | 457 | N256A | L457L |
| 257 | 458 | I257F | D458D |
| 258 | 492 | V258V | R492R |
| 259 | 501 | V259V | K501K |
| 260 | 509 | Q260Q | K509H |
| 324 | 510 | P324N | N510T |
| 330 | 511 | D330E | G511G |
| 341 | 512 | R341V | D512Q |
| 408 | 513 | F408P | S513H |
| 409 | 514 | Q409Q | P514D |
| 410 | 515 | Q410Q | I515L |
| 411 | 516 | W411W | T516V |
| 412 | 517 | S412V | V517I |
| 413 | | L413L | |

1. A chimeric norovirus capsid protein comprising one or more of the following amino acid substitutions in any combination, wherein the numbering is based on the reference amino acid sequence of a norovirus strain GII.3 identified as SEQ ID NO:1:
a) amino acid residues R341V and S412V (Epitope F);
b) amino acid residues D512Q, S513H, and P514D (Epitope I); and/or
c) amino acid residues I234V, P324N, and D330E (NERK).

2. The chimeric norovirus capsid protein of claim 1, comprising the amino acid sequence of SEQ ID NO:1 (GII.3), wherein said amino acid sequence comprises one or more of the following amino acid substitutions in any combination:
a) amino acid residues R341V and S412V (Epitope F);
b) amino acid residues D512Q, S513H, and P514D (Epitope I); and/or
c) amino acid residues I234V, P324N, and D330E (NERK).

3. The chimeric norovirus capsid protein of claim 1, further comprising one or more of the following amino acid substitutions in any combination:
T254S, E255G, N256A, I257F, F408P, K509H, and/or N510T.

4. The chimeric norovirus capsid protein of claim 1, comprising the amino acid sequence of any one of SEQ ID NO:2-14.

5-16. (canceled)

17. The chimeric norovirus of claim 1, further comprising one or more of the following amino acid substitutions in any combination:
D247E, S248K, H250F, S252G, Q445T, G455M, V456N, I515L, T516V and/or V517I.

18. The chimeric norovirus capsid protein of claim 17, comprising the amino acid sequence of SEQ ID NO:15 (GII.3/GII.4 P8).

19. A synthetic backbone molecule comprising a set of amino acid residues that form a norovirus conformation epitope, comprising the following set of amino acid substitutions, wherein the numbering is based on the reference amino acid sequence of a norovirus capsid protein of a norovirus strain GII.3 identified as SEQ ID NO:1:
a) amino acid residues R341V and S412V (Epitope F);
b) amino acid residues D512Q, S513H, and P514D (Epitope I); and/or
c) amino acid residues I234V, P324N, and D330E (NERK),
wherein the synthetic backbone molecule is not a norovirus capsid protein.

20. The synthetic backbone molecule of claim 19, further comprising one or more of the following amino acid substitutions in any combination:
D247E, S248K, H250F, S252G, T254S, E255G, N256A, I257F, F408P, Q445T, G455M, V456N K509H, N510T, and/or I515L, T516V and/or V517I.

21. A norovirus P particle comprising a set of amino acid residues that form a norovirus conformation epitope, comprising one or more of the following amino acid substitutions in any combination, wherein the numbering is based on the reference amino acid sequence of a norovirus capsid protein of a norovirus strain GII.3 identified as SEQ ID NO:1:
a) amino acid residues R341V and S412V (Epitope F);
b) amino acid residues D512Q, S513H, and P514D (Epitope I); and/or
c) amino acid residues I234V, P324N, and D330E (NERK),
wherein the synthetic backbone molecule is not a norovirus capsid protein.

22. The norovirus P particle of claim 21, further comprising one or more of the following amino acid substitutions in any combination:
D247E, S248K, H250F, S252G, T254S, E255G, N256A, I257F, F408P, Q445T, G455M, V456N K509H, N510T, and/or I515L, T516V and/or V517I.

23. A synthetic nanoparticle and/or scaffold immunogen comprising amino acid residues 224 through 529 of the chimeric norovirus capsid protein of claim 1, wherein the numbering is based on the reference amino acid sequence of a norovirus capsid protein of a norovirus strain GII.3 identified as SEQ ID NO:1.

24. An isolated nucleic acid molecule encoding the chimeric norovirus capsid protein of claim 1.

25. A virus replicon particle (VRP) comprising the nucleic acid molecule of claim 24.

26. A vector comprising the nucleic acid molecule of claim 25.

27. (canceled)

28. A virus like particle (VLP) comprising the capsid protein of claim 1.

29. (canceled)

30. A composition comprising the chimeric norovirus capsid protein of claim 1 in a pharmaceutically acceptable carrier.

31. A method of producing an immune response to a norovirus in a subject, comprising administering to the subject an effective amount of the chimeric norovirus capsid protein of claim 1.

32. A method of treating a norovirus infection in a subject, comprising administering to the subject an effective amount of the chimeric norovirus capsid protein of claim 1.

33. (canceled)

34. A method of reducing the risk of developing a disorder associated with norovirus infection in a subject, comprising administering to the subject an effective amount of the chimeric norovirus capsid protein of claim 1.

35. A method of protecting a subject from the effects of norovirus infection, comprising administering to the subject an effective amount of the chimeric norovirus capsid protein of claim 1.

36-39. (canceled)
